(19) United States
(12) Patent Application Publication
(10) Pub. No.: US 2025/0381149 A1
(43) Pub. Date: Dec. 18, 2025

(54) CATIONIC AMPHIPHILIC COMPOUND-BASED NANOPARTICLE COMPOSITIONS

(71) Applicant: Universiteit Gent, Gent (BE)

(72) Inventors: Koen Raemdonck, Gent (BE); Stefaan De Smedt, Gent (BE); Bram Bogaert, Gent (BE)

(73) Assignee: Universiteit Gent, Gent (BE)

(21) Appl. No.: 18/879,148

(22) PCT Filed: Jun. 30, 2023
(86) PCT No.: PCT/EP2023/068031

§ 371 Date: Dec. 26, 2024

(30) Foreign Application Priority Data
Jun. 30, 2022 (EP) 22182324.8

(51) Int. Cl. A61K 9/51; A61K 31/713

(52) U.S. Cl. CPC A61K 9/5123; A61K 31/713

(57) ABSTRACT

The present invention relates to a method and compositions for optimized cytosolic delivery of active agents, in particular nucleic acids, using a specific class of cationic amphiphilic compounds. The method and compositions of the invention enhance intracellular release of the agents and can be used for the treatment of various disorders.

### FIELD OF THE INVENTION

The present invention relates to a method and compositions for optimized cytosolic delivery of active agents, in particular nucleic acids, using a specific class of cationic amphiphilic compounds. The method and compositions of the invention enhance intracellular release of the agents and can be used for the treatment of various disorders.

### BACKGROUND TO THE INVENTION

Nucleic acid therapeutics are an emerging class of drugs that address diseases at the genomic and/or transcriptomic level. For example, small interfering RNA (siRNA) and messenger RNA (mRNA) both enable regulation of intracellular protein concentrations. Following cytosolic delivery, siRNAs activate the RNA interference (RNAi) pathway, leading to sequence-specific silencing of genes at the post-transcriptional level, while delivery of in vitro transcribed mRNA can drive expression of therapeutic proteins and antigens. To overcome the many extra- and intracellular barriers upon in vivo administration, including nuclease degradation, tissue distribution and delivery across cellular membranes, RNA therapeutics are typically encapsulated in synthetic nanoparticles (NPs). Among the various NPs under investigation, lipid nanoparticles (LNPs) currently are the preferred carrier material for RNA delivery. LNPs generally contain one or more helper lipids (e.g. DOPE, DSPC, cholesterol, etc.) and a cationic or ionizable lipid, the latter being responsible for electrostatic complexation of the oppositely charged RNA and subsequent endosomal escape.

Various types of cationic lipids, ionizable lipids and lipid-like molecules have been designed with diverging physicochemical properties for LNP formulation. However, despite the great promise for e.g. RNA therapeutics, even for state-of-the-art LNPs, intracellular delivery often remains inefficient, with only 1-4% of the endocytosed RNA dose actually escaping the endosomal confinement to reach the cytosol. In addition, besides facilitating RNA encapsulation and cellular delivery, synthetic lipids do not always have desirable biological activity. Indeed, cellular toxicity and immunogenicity are major potential drawbacks associated with the use of cationic LNPs, especially when repeated administration is required. As such, alternative materials should be considered that enable sufficient cytosolic release of the encapsulated RNA with acceptable cellular toxicity.

Recent studies have shown that widely used cationic amphiphilic drugs (CADs) can be repurposed as small nucleic acid delivery enhancers (Joris et al., 2018). CADs are pharmacologically diverse compounds (e.g. antidepressants, antihistamines, antihypertensives) that tend to accumulate in acidified lysosomes given their amphiphilic and weak basic properties, leading to functional acid sphingomyelinase (ASM) inhibition. This so-called acquired lysosomal storage disease phenotype leads to transient lysosomal membrane permeabilization (LMP), which allows the passage of small nucleic acid therapeutics. However, it was also observed that the CAD-induced pore size in the lysosomal limiting membrane does not allow endolysosomal escape of larger RNA cargo, precluding the extrapolation of this drug delivery concept to mRNA therapeutics (Van de Vyver et al., 2020).

WO2017/34991 discloses therapeutic polymeric nanoparticles that include a nucleic acid and a hydrophobic counterion comprising amongst others CADs. WO2017/34991 however is silent on lipid based nanoparticles.

Kulkarni et al. described the formulation of lipid based nanoparticles to co-encapsulate the hydrophilic weak basic drug amphotericin B and small interfering RNA (siRNA). However, due to the relatively low clog P value (˜-0.66, ALOGPS, go.drugbank.com), this drug is not considered a CAD. Moreover, in this formulation the amphotericin B (1) is not employed to replace the ionizable/cationic lipid in the LNP formulation, (2) is therefore not employed for siRNA complexation and (3) is not employed as a structural component of the LNP. Alternatively, Zhang et al. describe the formulation of LNPs in which part of the cholesterol fraction is substituted by the neutral anti-inflammatory corticosteroid dexamethasone. However, this work demonstrated that the LNP's transfection efficiency with increasing dexamethasone content was substantially reduced.

In the present invention, we have identified that specific cationic amphiphilic compounds (CACs) and e.g. nucleic acids can be co-encapsulated in the same nanoparticle formulation and maintain or improve their biological activity, providing opportunities for drug combination therapy. Moreover, in the present invention, we have identified a lipid nanoparticle formulation in which cationic amphiphilic compounds can fully substitute the ionizable cationic lipid fraction of a LNP formulation with maintained nucleic acid delivery capacity.

### SUMMARY OF THE INVENTION

The present invention relates to a method, compositions and uses thereof, for optimized cytosolic delivery of active agents and/or for use in combination therapy. The composition comprises, consists essentially of, or consists of a lipid-based nanocarrier, an active agent and at least one amphiphilic compound, in particular a cationic amphiphilic compound (CAC), more in particular a cationic amphiphilic drug (CAD); even more in particular a CAC or CAD comprising at least one cyclic moiety.

In a particular embodiment, the present invention provides a nanoparticle comprising at least one lipid, at least one cationic amphiphilic compound (CAC) having a c log P value of less than 10, and at least one nucleic acid molecule.

In another particular embodiment, the present invention provides a nanoparticle comprising at least one lipid, at least one cationic amphiphilic compound (CAC) having a c log P value of less than 10, and at least one nucleic acid molecule; wherein said CAC comprises at least one cyclic moiety.

In one embodiment, the CAC has a c log P value of at least 1 and/or the CAC comprises one or more basic amines and/or the CAC comprises at least one cyclic moiety.

In another embodiment, the nanocarrier is a nanoparticle, in particular a nanoparticle comprising, consisting essentially of, or consisting of at least one active agent, at least one lipid component and at least one CAC. The nanocarrier of the present invention is particularly useful for delivering an agent, such as a membrane-impermeable agent, into the cytosol of a cell by release of the agent from the endosomal and/or endolysosomal compartment. The agent can be a diagnostic or therapeutic agent, in particular a nucleic acid; more in particular a nucleic acid selected from the group consisting of DNA, RNA, hybrids thereof, RNAi-inducing agents, RNAi agents, antisense RNAs, ribozymes, catalytic DNA, circular RNA, guide RNA, RNAs that induce triple helix formation, aptamers, and vectors; even more in particular said RNA is selected from the group consisting of an antisense compound, messenger RNA (mRNA), short interfering nucleic acid (siNA), small interfering RNA (siRNA), asymmetrical interfering RNA (aiRNA), double stranded RNA (dsRNA), micro-RNA (miRNA), small nucleolar RNA (sno-RNA), Piwi-interacting RNA (piRNA), non-coding RNA (ncRNA) and short hairpin RNA (shRNA).

In addition, it was shown that the selected CAC retains its biological activity making the nanocarrier useful for combination therapies.

In particular, the CAC of the invention has a lipid-like structure. More specific, the CAC comprises at least one lipid selected from the group consisting of an ionizable lipid, cationic lipid, a phospholipid, a sterol, a PEGylated lipid, a sphingolipid (such as sphingomyelin), lysolipid, mixed acyl lipid, ether lipid, ester lipid, oxidized lipid, cholesterol lipid, neutral lipid, zwitterionic lipid, charged lipid, sterol analogue, sterol-modified lipid, natural lipid, glycosylated lipid, pH-sensitive lipid, isoprenoids, bacterial lipid, plant lipid, bioactive lipid, lipid adjuvants, coenzyme A modified lipid, photo switchable lipids, click lipids, bile lipid, headgroup-modified lipid, fatty acid modified lipids, inverted headgroup lipid, polymer-conjugated lipid, polymerizable lipid, stabilizing lipid, and any combination thereof.

In a very specific embodiment, said at least one lipid is a cationic lipid; in particular wherein the cationic or ionisable lipid is present in an amount about 5 to about 75 mole percent.

In yet a further embodiment, the nanoparticle of the invention comprises:
a) about 5 to about 75 mole percent of the cationic amphiphilic compound;
b) wt/wt ratio of the lipid components to the nucleic acid of between 5:1 to about 15:1, such as about 10:1; and
c) about 5 to about 75 mole percent of said at least one lipid.
d) optionally about 5 to about 75 mole percent of a cationic lipid.

In another embodiment, said at least one lipid (cf. (c)) is selected from the group consisting of an ionizable lipid, a cationic lipid, a phospholipid, a sterol, a PEGylated lipid, a sphingolipid (such as sphingomyelin), lysolipid, mixed acyl lipid, ether lipid, ester lipid, oxidized lipid, cholesterol lipid, neutral lipid, zwitterionic lipid, charged lipid, sterol analogue, sterol-modified lipid, natural lipid, glycosylated lipid, pH-sensitive lipid, isoprenoids, bacterial lipid, plant lipid, bioactive lipid, lipid adjuvants, coenzyme A modified lipid, photo switchable lipids, click lipids, bile lipid, headgroup-modified lipid, fatty acid modified lipids, inverted headgroup lipid, polymer-conjugated lipid, polymerizable lipid, stabilizing lipid, and any combination thereof.

In a particular embodiment, said CAC is used to fully replace the ionizable or cationic lipid as typically present in a lipid nanoparticle (LNP); accordingly in said embodiment, the nanoparticle does not comprise an ionisable or cationic lipid. These nanoparticles are also referred to herein as CADosomes.

In another embodiment, said CAC is used in combination with an ionizable or cationic lipid, and in such instances replaces part of the other lipids (such as part of the sterol component) typically used in a lipid nanoparticle. These nanoparticles are also referred to herein as CAD-LNPs.

In a specific embodiment said CAC is characterized by one or more of the following features:
c log P<10; in particular between 1 and 10, such as between 2 and 8; in particular between 3 and 6
comprising at least one cyclic moiety
molecular weight of less than 1000 g/mol, such as between 100 and 900 g/mol;
alternatively between 200 and 800 g/mol; alternatively between 300 and 500 g/mol;
comprising one or more basic amines of which the conjugated acid has a pKa (also indicated as pKa1) of at least 5, 6, 7 or higher (up to 10, 11, 12 or 13). More specifically, the compounds have a pKa of 5 or more, even more specific of 6 or more.
contains an open (i.e. linear) C-chain of less than 10 C-atoms; this C-chain cannot be interrupted by heteroatoms (e.g. N or S or O). The C-chain can be fully saturated or (partially) unsaturated. Every C-atom in this C-chain can be substituted with other functional groups.

In a specific embodiment, said CAC is a tricyclic compound.

These tricyclic compounds are particularly suitable for the preparation of CADosomes using the 2-step approach as detailed in the examples part.

In another particular embodiment, said CAC is represented by formula I

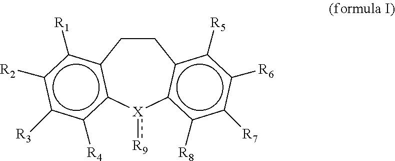

wherein
R₁, R₂, R₃, R₄, R₅, R₆, R₇ and R₈ are each independently selected from the group consisting of —H, —C₁₋₆alkyl and -halo;
R₉ is selected from group consisting of —(C₁₋₈alkyl)NR₁₀R₁₁, -Het₁,
R₁₀ and R₁₁ are each independently selected from the group consisting of —H and C₁₋₆alkyl;
Het₁ is a 5 or 6-membered heterocycle having from 1 to 3 heteroatoms selected from N, O and S,
X is selected from C, CH and N;
[symbol] represents a single or double bond, wherein when X is C, then [symbol] represents a double bond.

In a very specific embodiment said CAC is selected from the group consisting of:

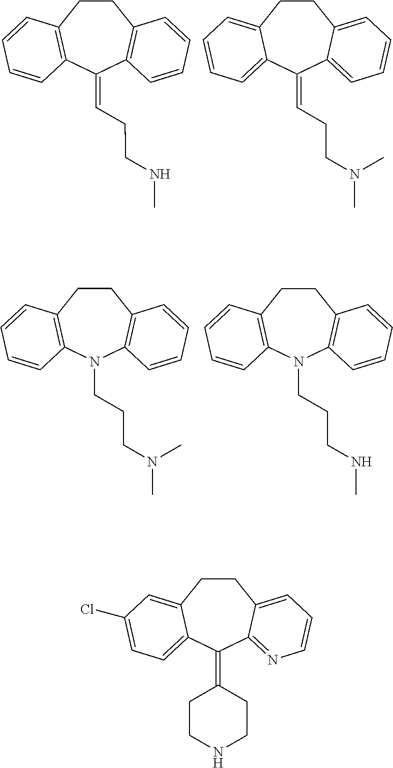

Other particularly suitable compounds are defined in the tables below.

In a further embodiment, the nanocarrier of the present invention is used in human or veterinary medicine, in particular in a method of delivering an agent into the cytosol of a cell by in vitro, ex vivo or in vivo application and/or in combination therapies. Furthermore, the present invention provides a method for delivery of an active agent across a cell membrane, said method comprising contacting cells with a nanoparticle as defined herein.

### BRIEF DESCRIPTION OF THE DRAWINGS

With specific reference to the figures, it is to be noted that the particulars shown are by way of example and for purposes of illustrative discussion of the different embodiments of the present invention. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings make it apparent to those skilled in the art how the several forms of the invention may be embodied in practice.

FIG. 1. Physicochemical characterization of mRNA CADosomes containing nortriptyline (NT)-DOPE (A) Schematic representation of NT-DOPE mRNA CADosomes, produced with vesicles obtained via an ethanol dilution (ED) or lipid film hydration (LFH) method. (B) Representative transmission electron microscopy (TEM) image of enhanced green fluorescent protein-encoding messenger RNA (eGFP-mRNA) NT-DOPE CADosomes, prepared via ED. Scale bar corresponds to 200 nm. Dynamic light scattering data (hydrodynamic size, polydispersity index (PDI) and zeta potential) of NT-DOPE and DOTAP-DOPE vesicles, (C-E) prior to (n=14) and (F—H) after complexation with eGFP-mRNA (n=3). (I-J) Fluorescence correlation spectroscopy (FCS) analysis of CADosome mRNA complexation efficiency with different N/P ratios. Data are represented as mean±the standard error of the mean (SEM) for minimum three independent repeats (n≥3). Statistical analysis was performed using One Way Anova with Tukey Correction (** p≥0.01, *** p≤0.001).

FIG. 2. Evaluating nortriptyline (NT)-DOPE CADosomes for cytosolic delivery of eGFP-encoding mRNA in a HeLa cell line. (A-B) Evaluation of cellular uptake of NT-DOPE CADosomes with different N/P ratios, loaded with Cy5-labeled mRNA, in HeLa cells as analyzed via flow cytometry and expressed as Cy5+ cells and Cy5 relative mean fluorescence intensity (rMFI Cy5; normalized to non-treated control (NTC)), respectively. (C-D) eGFP expression 24 h after transfection with NT-DOPE CADosomes N/P 3, 6, 9 and 12, DOTAP-DOPE N/P 2 and negative controls (CTRL) complexing luciferase-encoding mRNA, expressed as percentage eGFP+HeLa cells and eGFP rMFI, respectively. (E-F) Transfection yield (i.e. eGFP expression normalized to intracellular mRNA dose) of NT-DOPE CADosomes N/P 9 compared to DOTAP-DOPE lipoplexes. Data are represented as mean±the standard error of the mean (SEM) for minimum three independent repeats (n≥3). Statistical analysis was performed using One Way Anova with Tukey Correction (ns p>0.05, * p≤0.05, ** p≤0.01, *** p<0.001, **** p<0.0001).

FIG. 3. Screening of different CADosomes for mRNA delivery in HeLa cells. (A) Molecular structure of CAD molecules which were capable of forming CADosomes in combination with DOPE (50:50 ratio). (B-C) Flow cytometry quantification of cellular uptake and eGFP-mRNA expression of AMI-DOPE, DSI-DOPE, IMI-DOPE and DES-DOPE compared to NT-DOPE CADosomes, loaded with Cy5-labeled or eGFP-encoding mRNA, respectively. The latter CADosomes were prepared via ethanol dilution (B) and lipid film hydration method (C) using optimal N/P ratios. Data are represented as mean±the standard error of the mean (SEM) for minimum three independent repeats (n≥3). Statistical analysis was performed using One Way Anova with Tukey Correction (ns p≥0.05). (rMFI=relative mean fluorescence intensity normalized to non-treated cells (NTC)).

FIG. 4. Evaluation of the pharmacological activity of nortriptyline following CADosome formulation using a Nanoluciferase Binary Technology (NanoBiT®) bioassay. HEK293T cells stably expressing two inactive luciferase split fragments (1 kDa SmBiT and 18 kDa LgBiT), coupled to the 5-HT2AR (serotonin 2A receptor) and the cytosolic protein p-arrestin 2 (βarr2), respectively 54-56. Binding of a receptor agonist, in this case LSD, results in βarr2 recruitment to the 5-HT2AR with the concomitant functional complementation of the enzyme, which can be monitored through luminescence read-out. Binding of a receptor antagonist, e.g. nortriptyline (NT), inhibits LSD-induced βarr2 recruitment and subsequent luciferase complementation. (A) Percentage 5-HT2AR activation induced by blank, free NT (30 and 100 μM) and different NT-DOPE CADosomes loaded with eGFP-mRNA, measured by calculating the normalized area under the curve (AUC) values of the receptor activation profiles. 1 μM LSD was added to all samples and luminescence was continuously monitored for 2 h. Data are represented as mean±the standard error of the mean (SEM) for minimum three independent repeats (n≥3). Statistical analysis was performed using One Way Anova with Tukey Correction (****p<0.0001). (B-C-D) One representative activation profile of blank, free NT 30 μM and mRNA NT-DOPE N/P 9, respectively.

FIG. 5. CADosome-mediated delivery of Cre-recombinase encoding mRNA (Cre-mRNA) in a HeLa reporter cell line, shifting from DsRed+ to eGFP+after Cre-recombinase mediated elimination of the DsRed stop-codon following successful delivery of Cre-encoding mRNA via CADosomes. (A) Percentage eGFP+ cells as analyzed via flow cytometry 24 h after transfection with NT-DOPE CADosomes N/P 9-12 and DOTAP-DOPE N/P 2. Data are represented as mean±the standard error of the mean (SEM) for minimum three independent repeats (n≥3). Statistical analysis was performed using One Way Anova with Tukey Correction (*** p<0.0001). (B) Representative flow cytometry dot-plots of non-treated cells (NTC) and NT-DOPE CADosomes N/P 9 or DOTAP-DOPE N/P 2 transfected HeLa reporter cells, respectively.

FIG. 6. Delivery of mRNA with NT-DOPE CADosomes in primary bovine corneal epithelial cells (PBCEC). The corneal epithelial layer was separated from corneal stroma using stainless tweezers and cultured for mRNA transfection. (A) Cell uptake of Cy5-mRNA via NT-DOPE CADosomes N/P 9-12 was significantly lower compared to cationic DOTAP-DOPE lipoplexes after 4 h incubation. (B-C) CADosomes outperformed DOTAP-DOPE lipoplexes, reaching a ten-fold higher transfection yield for delivery of eGFP-mRNA to hard-to-transfect PBCECs, as measured 24 h after administration. (D) Representative dot-plots of non-treated cells (NTC), eGFP-mRNA NT-DOPE CADosomes N/P 9 and eGFP-mRNA DOTAP-DOPE N/P 2 lipoplexes analyzed via flow cytometry. Data are represented as mean±the standard error of the mean (SEM) for minimum three independent repeats (n≥3). Statistical analysis was performed using One Way Anova with Tukey Correction (* p<0.05, ** p≤0.01, *** p<0.001, **** p<0.0001).

FIG. 7. mRNA NT-DOPE CADosomes outperformed state-of-the-art MC3 LNPs in primary bovine corneal epithelial cells (PBCEC). (A) Percentage eGFP expression and (B) relative MFI values were analyzed via flow cytometry 24 h after administration. mRNA NT-DOPE N/P 9 CADosome reached a twenty-fold higher rMFI value of eGFP mRNA compared to both MC3-DOPE and MC3-DSPC LNPs. Data are represented as mean±the standard error of the mean (SEM) for minimum three independent repeats (n≥3). Statistical analysis was performed using One Way Anova with Tukey Correction (* p<0.05, ** p≤0.01, ***p<0.001, ****p<0.0001).

FIG. 8. Representative transmission electron microscopy (TEM) images of eGFP encoding mRNA NT-DOPE CADosomes, prepared via ethanol dilution. Scale bar 200 nm.

FIG. 9. Cell viability measured via Cell-Titer-Glo®. (A) Evaluation of cell viability in HeLa cell line (B) and Primary Bovine Corneal Epithelial Cells (PBCEC). NT-DOPE N/P 9 was indicated as the most optimal N/P ratio, without increasing cell toxicity in HELA cells. Higher N/P ratios showed no increase in PBCECs cell toxicity. Data are represented as mean±the standard error of the mean (SEM) for minimum three independent repeats (n≥3). Statistical analysis was performed using One Way Anova with Tukey Correction (ns p≥0.05).

FIG. 10. Evaluation of the CADosomes pharmacological activity using Nanoluciferase Binary Technology (NanoBiT©). HEK 293T (Human Embryonic Kidney) cell Nanoluciferase Binary Technology (NanoBiT©) system was used, stably expressing two inactive split fragments, the 5-HT2AR (Serotonin 2A Receptor) and the cytosolic protein μ-arrestin 2 (βarr2). Binding of a receptor agonist, in this case LSD, results in βarr2 recruitment to the 5-HT2AR with the concomitant functional complementation of the enzyme, which can be monitored through the luminescence generated in the presence of the substrate. Binding of a receptor antagonist e.g. nortriptyline (NT) inhibits protein interactions and luminescence generation. One representative receptor activation profile of blank, free NT 30 M, free NT 100 M, and NT-DOPE CADosomes at varying N/P ratio are shown. ED: ethanol dilution; LFH: lipid film hydration; Blank: 10 nM LSD and 1 M LSD was added in separate wells and luminescence was continuously monitored for 2 h.

FIG. 11. Quantification of eGFP mRNA expression with NT-DOPE CADosomes with and without OptiMEM pre-treatment in a HeLa cell line. (A-B) An increase in relative-mean fluorescence intensity (rMFI) could be observed for NT-DOPE CADosomes N/P 9 with OptiMEM (OPT) pre-treatment, while no significant difference was seen for DOTAP-DOPE lipoplexes. The percentage of eGFP-positive cells slightly increased for both mRNA complexes. (C) Normalized rMFI signal (rMFI OptiMEM vs no OptiMEM pre-treatment) of NT-DOPE CADosomes N/P 9 increased almost two-fold in HeLa cells, outperforming DOTAP-DOPE. (D) Representative flow cytometry histogram of eGFP expression with NT-DOPE CADosomes N/P 9 as a function of OptiMEM treatment. Data are represented as mean±the standard error of the mean (SEM) for minimum three independent repeats (n≥3). Statistical analysis was performed using One Way Anova with Tukey Correction (** p<0.01, *** p<0.001, **** p<0.0001).

FIG. 12. Physicochemical characterization of siRNA MC3/CAD-LNPs. Stability of respectively (A) state-of-the-art MC3 LNPs, (B) MC3 containing 10 mol % nortriptyline (NT), (C) MC3 containing 25 mol % NT and (D) 38.5 mol % NT measured over time via dynamic light scattering (size and PDI). (E) Zeta potential of siRNA MC3/CAD-LNPs. Data are represented as mean±the standard error of the mean (SEM) for minimum three independent repeats (n≥3).

FIG. 13. Encapsulation efficiency of siRNA MC3/CAD-LNPs measured via Quant-iT™ RiboGreen® Assay (InvitroGen). siRNA encapsulation slightly decreased for siRNA MC3-NT LNPs after reducing cholesterol fractions, but still remained above 60%. Data are represented as mean±the standard error of the mean (SEM) for minimum three independent repeats (n≥3).

FIG. 14. Quantification of MC3-nortriptyline (NT)-LNP (50:10 mol %) eGFP-siRNA knockdown efficiency in H1299_eGFP cell line. (A) Evaluation of cellular uptake of MC3- and MC3-NT LNPs, loaded with Cy5-labeled siRNA, in H1299-eGFP cells as analyzed via flow cytometry and expressed as Cy5+ cells and Cy5 relative mean fluorescence intensity (rMFI Cy5; normalized to non-treated control (NTC)), respectively. (B) eGFP expression 48 h after transfection with MC3- and MC3-NT LNP encapsulating eGFP-siRNA, expressed as percentage eGFP+H1299-eGFP cells. Data are represented as mean±the standard error of the mean (SEM) for minimum three independent repeats (n≥3). Statistical analysis was performed using One Way Anova with Tukey Correction (ns p>0.05).

FIG. 15. Quantification of MC3-nortriptyline (NT)-LNP (50:25 mol %) eGFP-siRNA knockdown efficiency in H1299_eGFP cell line. (A) Evaluation of cellular uptake of MC3- and MC3-NT LNPs, loaded with Cy5-labeled siRNA, in H1299-eGFP cells as analyzed via flow cytometry and expressed as Cy5+ cells and Cy5 relative mean fluorescence intensity (rMFI Cy5; normalized to non-treated control (NTC)), respectively. (B) eGFP expression 48 h after transfection with MC3- and MC3-NT LNP encapsulating eGFP-siRNA, expressed as percentage eGFP+H1299-eGFP cells. Data are represented as mean±the standard error of the mean (SEM) for minimum three independent repeats (n≥3). Statistical analysis was performed using One Way Anova with Tukey Correction (ns p>0.05).

FIG. 16. Quantification of MC3-nortriptyline (NT)-LNP (50:38.5 mol %) eGFP-siRNA knockdown efficiency in H1299_eGFP cell line. (A) Evaluation of cellular uptake of MC3- and MC3-NT LNPs, loaded with Cy5-labeled siRNA, in H1299-eGFP cells as analyzed via flow cytometry and expressed as Cy5+ cells and Cy5 relative mean fluorescence intensity (rMFI Cy5; normalized to non-treated control (NTC)), respectively. (B) eGFP expression 48 h after transfection with MC3- and MC3-NT LNP encapsulating eGFP-siRNA, expressed as percentage eGFP+H1299-eGFP cells. Data are represented as mean±the standard error of the mean (SEM) for minimum three independent repeats (n≥3). Statistical analysis was performed using One Way Anova with Tukey Correction (ns p>0.05).

FIG. 17. Screening different CAD molecules for siRNA CAD-LNP formation and cytosolic delivery in a H1299-eGFP cell line. (a) Schematic illustration of the selected CAD molecules, fluvoxamine (Fluv), fluoxetine (Fluox) and desloratadine (DES) with different molecular structure to form siRNA-loaded CAD-LNPs with reduced cholesterol fraction. (b) Encapsulation efficiency of siRNA-eGFP MC3-Fluv, MC3-Fluox and MC3-DES (50:25) LNPs, measured by the Quant-iT™ RiboGreen® RNA assay. (c-d) Dynamic light scattering data (hydrodynamic size, polydispersity index (PDI) and zeta potential) of MC3-Fluv, MC3-Fluox and MC3-DES (50:25) LNPs. Stability was analyzed after dialysis and storage in PBS at 4° C. up to 10 or 20 weeks after production. (e) Cellular uptake and (f) enhanced green fluorescent protein (eGFP) silencing of the MC3-Fluv, MC3-Fluox and MC3-DES (50:25) LNPs in a H1299-eGFP lung epithelial cell line at different siRNA concentrations. siRNA-eGFP MC3-Fluv and MC3-Fluox (50:25) outperformed the state-of-the-art MC3 LNPs in eGFP knockdown efficiency, while no significant difference was observed for MC3-DES (50:25). Data are represented as mean±the standard error of the mean (SEM) for minimum three independent repeats (n≥3). Statistical analysis was performed using One Way Anova with Tukey Correction (ns p>0.05, * p≤0.05, ** p≤0.01, ***p<0.001, **** p≤0.0001). Illustration created with BioRender.com.

FIG. 18. In vivo biodistribution of Cy5-siRNA loaded CAD-LNPs analyzed 24 h and 48 h after intranasal administration to C₅₇BL/6 mice. (a) Schematic illustration of the in vivo biodistribution procedure. (b) Cellular uptake of Cy5-siRNA MC3, MC3-NT (50:25) and MC3-Fluox (50:25) LNPs in macrophages and neutrophils, as measured via flow cytometry after bronchoalveolar lavage (BAL). (c) Flow cytometric analysis of macrophages, epithelial—and endothelial cells of a single cell digest of the lung tissue. All mice received a fixed Cy5-siRNA dose of 10 μg intranasally. Data are represented as mean±the standard error of the mean (SEM) (n=5). Statistical analysis was performed using One Way Anova with Tukey Correction (* p<0.05, ** p≤0.01, *** p<0.001, **** p<0.0001). Illustration created with BioRender.com.

FIG. 19. Physicochemical characterization of mRNA-loaded nortriptyline (NT)-CADosomes produced via microfluidic NanoAssemblr SPARK™ device. Formulations contained Nortriptyline (NT), DOPE, cholesterol and DMG-PEG in different mole ratios (N/P 10). (A-B) Evaluation of hydrodynamic diameter (Z-Average diameter), PDI and zeta potential, measured via Dynamic Light Scattering (DLS). Higher cholesterol content resulted in CADosomes with higher Z-Average and lower zeta potential. (C) Encapsulation efficiency of mRNA CADosomes measured via Quant-iT™ RiboGreen® Assay (InvitroGen). mRNA encapsulation efficiency slightly decreased with increasing cholesterol fractions. Data are represented as mean±the standard error of the mean (SEM) for minimum three independent repeats (n≥3).

FIG. 20. Evaluation of mRNA-loaded nortriptyline (NT)-CADosomes produced via microfluidic NanoAssemblr SPARK™ device for cytosolic delivery of eGFP-encoding mRNA (100 ng/well) in a HeLa cell line. (A-B) Evaluation of cellular uptake of NT-DOPE-CHOL-DMG-PEG with different mole ratios, loaded with DiD-dye. HeLa cells were analyzed via flow cytometry and expressed as DiD+ cells and DiD relative mean fluorescence intensity (rMFI DiD; normalized to non-treated control (NTC)), respectively. (C-D) eGFP expression 24 h after transfection with mRNA CADosomes produced via microfluidic NanoAssemblr SPARK™ device, expressed as percentage eGFP+HeLa cells and eGFP rMFI, respectively. CADosomes with less than 50% eGFP encapsulated mRNA were excluded from this experiment. Data are represented as mean±the standard error of the mean (SEM) for minimum two independent repeats (n≥2).

FIG. 21. Physicochemical characterization of mRNA-loaded CADosomes produced via microfluidic NanoAssemblr SPARK™ device using different CAD molecules, i.e. loperamide HCl (LOP), verapamil HCl (VER), ketotifen fumarate (KET), epinastine HCl (EPI), fluoxetine HCl (FLUOX), fluvoxamine maleate (FLUV), nortriptyline HCl (NT). Besides the CAD molecule, formulations contained DOPE, cholesterol and DMG-PEG (50:38.5:10:1.5 mole ratio; N/P 6). (A-B) Evaluation of hydrodynamic diameter (Z-Average diameter), PDI and zeta potential, measured via Dynamic Light Scattering (DLS). (C) Encapsulation efficiency of mRNA CADosomes measured via Quant-iT™ RiboGreen® Assay (InvitroGen).

FIG. 22. In vitro mRNA delivery with spiked mRNA CADosomes containing the long acting beta agonist vilanterol. (a) table showing the molar compositions of the different LNP formulations, prepared via the established manual vortex mixing method. (b) Encapsulation efficiency of the LNP formulations via the Quant-iT™ RiboGreen® RNA assay. (c) eGFP relative mean fluorescence intensities normalized to equal LNPs encapsuling luciferase mRNA (rMFI) in BEAS-2B cells. (d) Relative percentage of eGFP MFI of spiked CAD-LNP formulations compared to parent LNPs containing only the cationic ionizable lipid. Data are represented as mean±the standard error of the mean (SEM) (n=x, x independent transfection with y LNP batches). Statistical analysis was performed using One Way Anova with Tukey Correction (* p 15≤0.05, ** p≤0.01).

FIG. 23. In vitro mRNA delivery with spiked mRNA CADosomes containing the long acting beta agonist salmeterol. (a) Table showing the molar compositions of the different LNP formulations, prepared via the established manual vortex mixing method. (b) Encapsulation efficiency of the LNP formulations via the Quant-iT™ RiboGreen® RNA assay. (c) eGFP relative mean fluorescence intensities normalized to equal LNPs encapsuling luciferase mRNA (rMFI) in BEAS-2B cells. (d) Relative percentage of eGFP MFI of spiked CAD-LNP formulations compared to parent LNPs containing only the cationic ionizable lipid. Data are represented as mean±the standard error of the mean (SEM) (n=x, x independent transfection with y LNP batches). Statistical analysis was performed using One Way Anova with Tukey Correction (* p 0.05, ** p≤0.01).

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. As used in the specification and the appended claims, the singular forms “a”, “an”, and “the” include plural referents unless the context clearly dictates otherwise. By way of example, “a compound” means one compound or more than one compound. Throughout the description and claims of this specification the word “comprise” and other forms of the word, such as “comprising” and “comprises,” means including but not limited to, and is not intended to exclude, for example, other additives, components, integers, or steps. The term “consisting essentially of” or “consists essentially of” means that e.g. a product or method must contain the listed compounds, ingredient(s), or steps and may also contain small amounts (for example up to 5% by weight, or up to 1% or 0.1% by weight) of other ingredient(s), compounds, or steps provided that any additional ingredients, compounds, or steps do not affect the essential properties of the respective product or method. The terms described above and others used in the specification are well understood to those in the art. All references, and teachings specifically referred to, cited in the present specification are hereby incorporated by reference in their entirety.

The current invention is directed to a specific selection of compounds that can be incorporated in a nanocarrier and that are used to enhance or facilitate delivery of therapeutic, biologically active or diagnostic agents into cells, more in particular for the cellular delivery of membrane-impermeable molecules in general. In addition, the compounds can also be considered for drug combination therapy.

A selection of amphiphilic compounds was identified that can be used as both structural and functional components of a nanocarrier, in particular lipid-based nanoparticles, more in particular lipid nanoparticles (LNPs) and lipoplexes (LPXs), e.g. to partly or fully replace (potentially harmful) synthetic cationic or ionizable lipids and/or to promote nucleic acid delivery efficiency.

In addition, incorporating both adjuvants and nucleic acids into lipid-based nanoparticles should enable to merge the therapeutic activities of both drugs in a single formulation. These compounds are identified herein as cationic amphiphilic compounds (CACs) including cationic amphiphilic drugs (CADs).

Accordingly, in a first aspect, the present invention provides a nanoparticle comprising at least one compound, in particular a cationic amphiphilic compound (CAC), the nanoparticle being effective in endocytosis. It should also be appreciated that endocytosis may also include any type of receptor-mediated endocytosis or other endocytic uptake pathways. For example, endocytosis may involve macropinocytosis, clathrin-dependent or clathrin-independent endocytosis, for instance endocytosis via caveolae, the invaginations in plasma membranes that have the potential to undergo endocytosis.

In one embodiment, the compound is an endo-lysosomal disrupting agent, i.e. any molecule, ion, or compound that is capable of, for example, substantially avoiding and/or limiting the processes of an endosome or lysosome of a cell. As contemplated herein, endocytosis is a pathway into the cell. In the process of endocytosis, endosomes are formed. Endo-lysosomal agents are able to break the membrane of the endosome and escape transport to a lysosome for destruction. Such agents may include compounds having mechanisms of action related to endosome or lysosome maturation, processing, and/or recycling.

In a particular embodiment of the present invention a specific selection of cationic amphiphilic compounds (CACs), including cationic amphiphilic drugs (CADs) or salts thereof, are incorporated in a nanoparticle. CADs are a very diverse class of small molecular pharmacological agents that are structurally characterized by a hydrophobic group (e.g. including aromatic rings) and a polar group containing a basic amine. As many CADs are widely used drugs (e.g. antihistamines, antidepressants, antipsychotics, antihypertensives, . . . ) with a well-documented safety profile and bioactivity, their repurposing as both functional and structural components in nanoparticles offers the prospect of efficient and non-toxic nucleic acid delivery while safeguarding clinical translatability. Typically, CADs have a moderate c log P value compared to conventional cationic/ionizable lipids and have a molecular weight below 1000 g/mol (e.g. ranging from 100 to 900, or from 200 to 800 g/mol) and can thus be considered as small molecules. In particular, the amphiphilic compounds of the present invention are cationic amphiphilic compounds having a c log P value of at least 1, preferably at least 2, more preferably at least 3, or higher (up to 7, 8, 9, or 10; such as e.g. a c log P of 2-10 , 2-9, 3-10, 3-9, 3-8, 3-7, 4-10 or 4-9). In a further embodiment, these compounds contain one or more basic amines of which the conjugated acid has a pKa (also indicated as pKa1) of at least 5, 6, 7 or higher (up to 10, 11, 12 or 13). More specifically, the compounds have a pKa of 5 or more, even more specific of 6 or more. Even more specifically, the CAC may comprise at least one cyclic moiety.

Such physicochemical properties can be calculated via dedicated software tools (e.g. ACD labs, Chemdraw Professional) and/or can be derived from (publicly available) chemical compound databases, in particular DrugBank or PubChem. The c log P is a calculated log P value (c log P), e.g. based on a fragment approach for c log P (octanol-water) prediction. Where a log P value is known for a particular compound, this value may be used instead of the calculated c log P value. Furthermore, the cationic amphiphilic compounds can comprise one or more basic amines.

As such, in one embodiment, the invention provides a lipid-based nanoparticle comprising at least one cationic amphiphilic compound (CAC) having a c log P value of less than 10, at least one lipid, and at least one nucleic acid molecule.

Accordingly, in a specific embodiment said CAC is characterized by one or more of the following features:
c log P<10; in particular between 1 and 10, such as between 2 and 8; in particular between 3 and 6
comprising at least one cyclic moiety
molecular weight of less than 1000 g/mol, such as between 100 and 900 g/mol;
alternatively between 200 and 800 g/mol; alternatively between 300 and 500 g/mol;
contains an open (i.e. linear) C-chain of less than 10 C-atoms; this C-chain cannot be interrupted by heteroatoms (e.g. N or S or O). The C-chain can be fully saturated or partially unsaturated. Every C-atom in this C-chain can be substituted with other functional groups.

More particular, in one embodiment the CAC is a tricyclic compound, which may be characterized by the presence of an aromatic tricyclic domain with a three carbon tail substituted with secondary- or tertiary methylated amine groups, or in the alternative a heterocyclic amine piperidine group. In addition, said tricyclic compound preferably contains a positive charge at physiological pH, i.e. pH 7.4. Moreover, CACs with a pKa of above 7.4 seem to be highly suitable for use in the present invention.

Accordingly in a specific embodiment, the CAC is represented by formula I

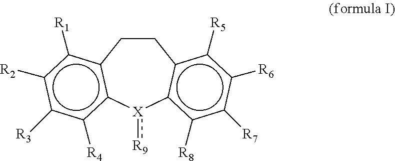

wherein
R₁, R₂, R₃, R₄, R₅, R₆, R₇ and R₈ are each independently selected from the group consisting of —H, —C₁₋₆alkyl and -halo;
R₉ is selected from group consisting of —(C₁₋₈alkyl)NR₁₀R₁₁, -Het₁,
R₁₀ and R₁₁ are each independently selected from the group consisting of —H and C₁₋₆alkyl;
Het₁ is a 5 or 6-membered heterocycle having from 1 to 3 heteroatoms selected from N, O and S,
X is selected from C, CH and N;
[symbol] represents a single or double bond, wherein when X is C, then [symbol] represents a double bond.

When describing the compounds of the invention, the terms used are to be construed in accordance with the following definitions, unless a context dictates otherwise:

The term “alkyl” by itself or as part of another substituent refers to a fully saturated hydrocarbon of Formula CₓH₂ₓ₋₁ wherein x is a number greater than or equal to 1. Generally, alkyl groups of this invention comprise from 1 to 6 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl, and its isomers (e.g. n-butyl, i-butyl and t-butyl); pentyl and its isomers, hexyl and its isomers. C₁-C₆ alkyl includes all linear, branched, or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopentyl, 2-, 3—, or 4-methylcyclopentyl, cyclopentylmethylene, and cyclohexyl.

The terms “heterocyclyl” or “heterocyclo” as used herein by itself or as part of another group refer to non-aromatic, fully saturated or partially unsaturated cyclic groups (for example, 5 to 6 member monocyclic ring systems) which have at least one heteroatom in a carbon atom-containing ring. The heterocyclic group typically contains 1, 2, or 3 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring system, where valence allows. Exemplary heterocyclic groups for example include piperidinyl.

The term “halo” or “halogen” as a group or part of a group is generic for fluoro, chloro, bromo, or iodo; in particular chloro.

Exemplary compounds particularly useful in the present invention are shown in Table 1.

In a particular embodiment the CAC is a CAD, and more specific nortriptyline hydrochloride (NT), amitriptyline hydrochloride (AMI), desipramine hydrochloride (DSI), imipramine hydrochloride (IMI), or desloratadine (DES), including combinations thereof.

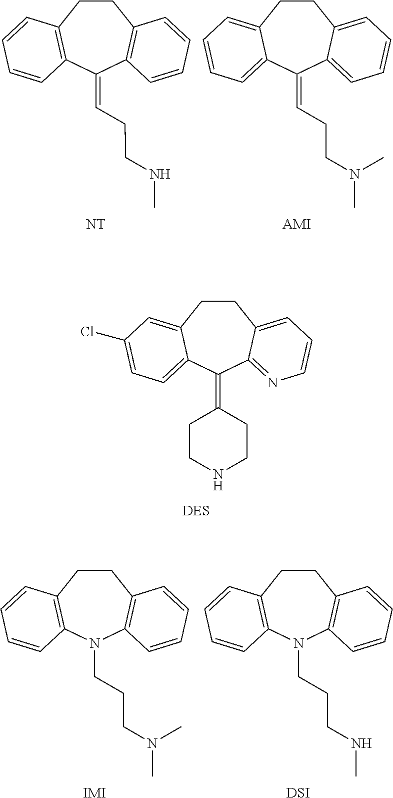

The c log P values shown in the table below were obtained from the public DrugBank database (Wishart DS, 2006, predicted with ChemAxon software, ChemAxon Ltd., Budapest, Hungary) and the pKa values shown are calculated via ACD/Labs (I-Lab 2.0—ilab.acdlabs.com). All compounds have a c log P>3.

TABLE 1
Adjuvant compounds that comply with the CAD definition (clogP > 3, pKa > 5).
(pKa1 = macroscopic pKa of the conjugated acid of the most basic amine. Structures
were obtained from JChem for Office (version 17.21.0.1797, ChemAxon Ltd., Budapest, Hungary).

| Compound name | Structure | clogP | pKa1 |
|---|---|---|---|
| Thiothixene | 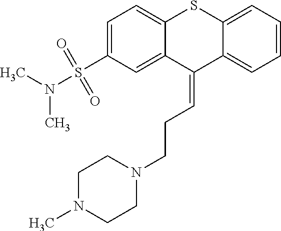 [SMILES: CN1CCN(CC/C=C2/c3ccccc3Sc3ccc(S(=O)(=O)N(C)C)cc32)CC1] | 3.36 | 8.16 |
| Thioridazine hydrochloride | 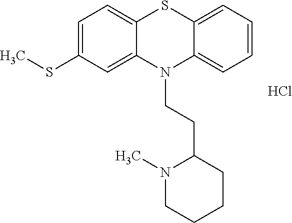 [SMILES: CSc1ccc2c(c1)N(CCC1CCCCN1C)c1ccccc1S2 ; Cl] | 5.47 | 8.93 |
| Desloratadine | 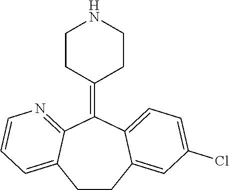 [SMILES: Clc1ccc2c(c1)CCc1cccnc1C2=C1CCNCC1] | 3.97 | 9.73 |
| Ketotifen | 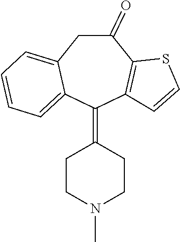 [SMILES: CN1CCC(=C2c3ccccc3CC(=O)c3sccc32)CC1] | 3.35 | 7.15 |
| Perphenazine | 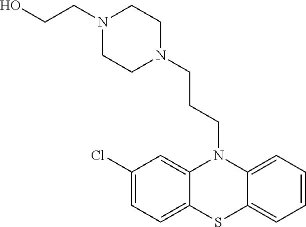 [SMILES: OCCN1CCN(CCCN2c3ccccc3Sc3ccc(Cl)cc32)CC1] | 3.69 | 7.81 |
| Loperamide hydrochloride | 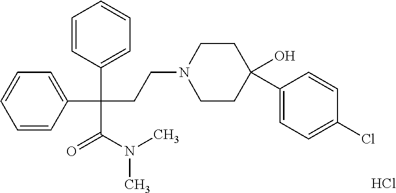 [SMILES: CN(C)C(=O)C(CCN1CCC(O)(c2ccc(Cl)cc2)CC1)(c1ccccc1)c1ccccc1 ; Cl] | 4.77 | 9.41 |
| Verapamil hydrochloride | 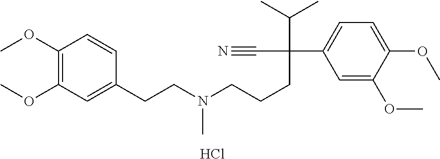 | 5.04 | 9.68 |
| Epinastine | 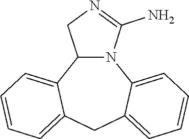 [SMILES: NC1=NCC2c3ccccc3Cc3ccccc3N12] | 3.13 | 9.31 |
| Clofazimine | 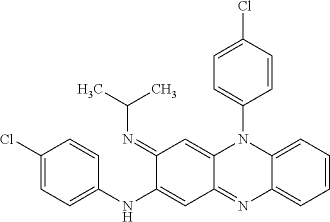 [SMILES: CC(C)/N=c1\cc2n(-c3ccc(Cl)cc3)c3ccccc3nc-2cc1Nc1ccc(Cl)cc1] | 7.30 | 6.63 |
| Clomipramine hydrochloride | 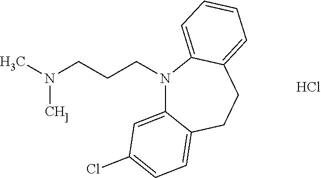 | 4.88 | 9.20 |
| Lofepramine | 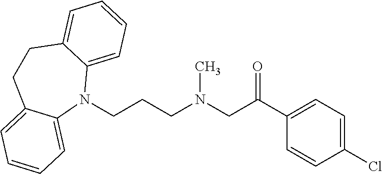 [SMILES: CN(CCCN1c2ccccc2CCc2ccccc21)CC(=O)c1ccc(Cl)cc1] | 6.11 | 6.53 |
| Trifluoperazine hydrochloride | 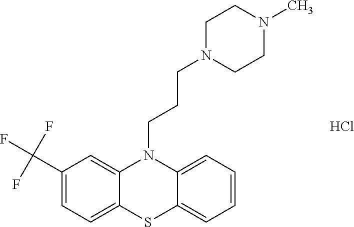 [SMILES: CN1CCN(CCCN2c3ccccc3Sc3ccc(C(F)(F)F)cc32)CC1 ; Cl] | 4.66 | 7.99 |
| Carvedilol | 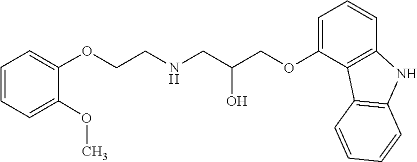 [SMILES: COc1ccccc1OCCNCC(O)COc1cccc2[nH]c3ccccc3c12] | 3.42 | 8.74 |
| SB 205607 dihydrobromide | 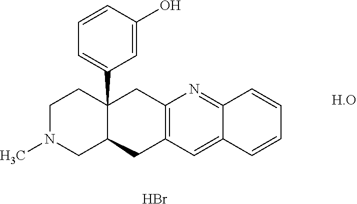 [SMILES: CN1CC[C@]2(c3cccc(O)c3)Cc3nc4ccccc4cc3C[C@H]2C1 ; Br] | 3.92 | 8.28 |
| Amitriptyline hydrochloride | 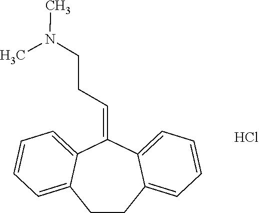 [SMILES: CN(C)CCC=C1c2ccccc2CCc2ccccc21 ; Cl] | 4.81 | 9.76 |
| Cyproheptadine hydrochloride | 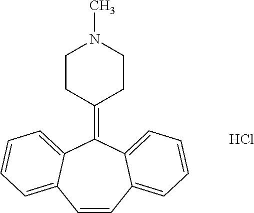 [SMILES: CN1CCC(=C2c3ccccc3C=Cc3ccccc32)CC1 ; Cl] | 4.38 | 8.05 |
| Rimcazole dihydrochloride | 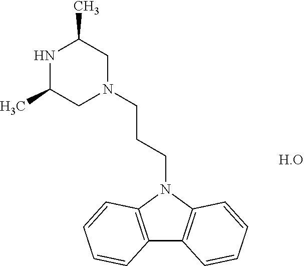 [SMILES: C[C@@H]1CN(CCCn2c3ccccc3c3ccccc32)C[C@H](C)N1] | 3.69 | 9.81 |
| Imipramine hydrochloride | 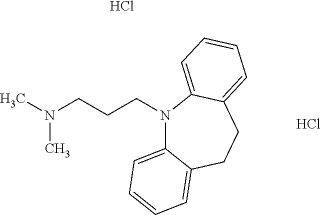 [SMILES: Cl ; CN(C)CCCN1c2ccccc2CCc2ccccc21 ; Cl] | 4.28 | 9.20 |
| Naltrindole hydrochloride hydrate | 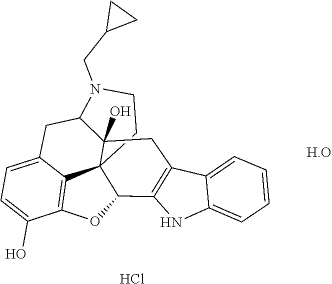 [SMILES: Oc1ccc2c3c1O[C@H]1c4[nH]c5ccccc5c4C[C@@]4(O)C(C2)N(CC2CC2)CC[C@]314 ; Cl] | 3.07 | 8.64 |
| Prochlorperazine maleate | 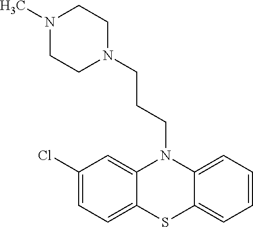 [SMILES: CN1CCN(CCCN2c3ccccc3Sc3ccc(Cl)cc32)CC1] | 4.38 | 7.99 |
| | 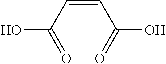 | | |
| Chlorpromazine hydrochloride | 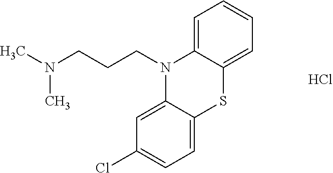 [SMILES: CN(C)CCCN1c2ccccc2Sc2ccc(Cl)cc21 ; Cl] | 4.54 | 9.20 |
| CGS 12066B dimaleate | 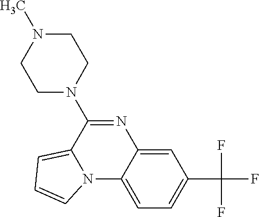 [SMILES: CN1CCN(c2nc3cc(C(F)(F)F)ccc3n3cccc23)CC1] | 3.22 | 7.61 |
| | 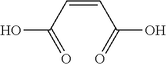 | | |
| Amoxapine | 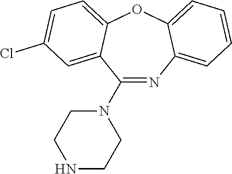 [SMILES: Clc1ccc2c(c1)C(N1CCNCC1)=Nc1ccccc1O2] | 3.08 | 8.83 |
| Desipramine hydrochloride | 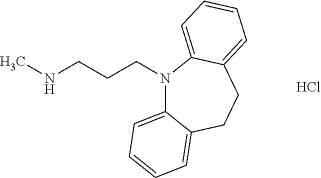 [SMILES: CNCCCN1c2ccccc2CCc2ccccc21 ; Cl] | 3.90 | 10.02 |
| Olanzapine | 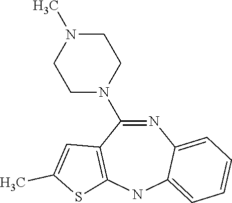 | 3.39 | 7.24 |
| | 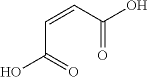 | | |
| Pizotyline maleate | 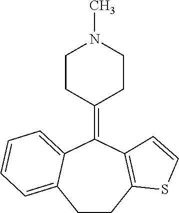 [SMILES: CN1CCC(=C2c3ccccc3CCc3sccc32)CC1] | 4.49 | 7.98 |
| Clozapine | 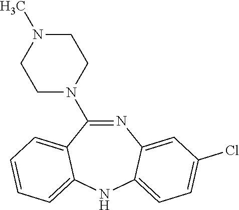 [SMILES: CN1CCN(C2=Nc3cc(Cl)ccc3Nc3ccccc32)CC1] | 3.40 | 7.35 |
| Promethazine hydrochloride | 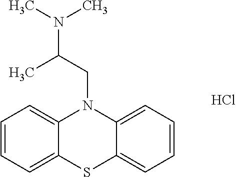 [SMILES: CC(CN1c2ccccc2Sc2ccccc21)N(C)C ; Cl] | 4.29 | 9.05 |
| Nortriptyline hydrochloride | 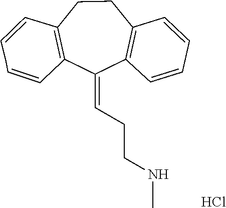 [SMILES: CNCC/C=C1/C2=C(C=CCC=C2)CCc2ccccc21 ; Cl] | 4.51 | 10.00 |
| Vilanterol | 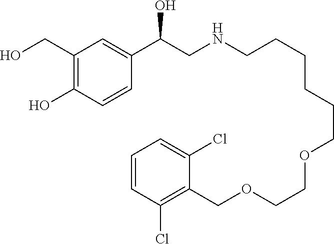 [SMILES: OCc1cc([C@@H](O)CNCCCCCCOCCOCc2c(Cl)cccc2Cl)ccc1O] | 3.61 | |
| Salmeterol | 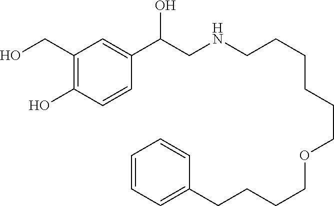 [SMILES: OCc1cc(C(O)CNCCCCCCOCCCCc2ccccc2)ccc1O] | 3.60 | |

Exemplary cationic amphiphilic compounds are tetrandrine, astemizole, terfenadine, ebastine, perhexaline, mepyramine, hydroxyzine, alimenazine, cyamemazine, dibucaine, propericiazine, thioproperazine, trihexyphenidyl, leelamine, ethyl lauryl arginatechl, promazine, tamoxifen, clomiphene, raloxifene, tamoxifen citrate, toremifene, clomiphene citrate, toremifene citrate, verapamil, diltiazem, amlodipine, nifedipine, nimodipine, felodipine, nicardipine, nisoldipine clevidipine, isradipine, trandolapril, desipramine, clomipramine, doxepin, amoxapine, trimipramine, protriptyline, amiodarone, sotalol, dronedarone, flecainide, procainamide, propafenone, quinidine, dofetilide, mexiletine, ibutilide, disopyramide, sertraline, escitalopram, citalopram, fluvoxamine, paroxetine hydrochloride, nefazodone, olanzapine, chlorcyclizine, amodiaquine, thioridazine, quinine, atovaquone/proguanil, atovaquone, fluoxetine, mefloquine, primaquine, quinacrine, quinidine, halofantrine, chloroquine, monensin, antrafenine, aripiprazole, bifeprunox, brexpiprazole, cariprazine, ciprofloxacin, dapiprazole, dropropizine, elopiprazole, etoperidone, itraconazole, ketoconazole, levodropropizine, mepiprazole, mianserin, naftopidil, nefazodone, niaprazine, oxypertine, posaconazole, trazodone, umespirone, urapidil, vesnarinone, lubazodone, acaprazine, batoprazine, bifeprunox, vortioxetine, vilazodone, tolpiprazole, sonepiprazole, pardoprunox, naphthylpiperazine, naluzotan, lorpiprazole, flesinoxan, fluprazine, flibanserin, ensaculin, enpiprazole, eltoprazine, elopiprazole, UNC 7938, sphingosine, dodecylimidazole, bafilomycin Al, quinolones, omeprazole, esomoprazole, pantoprazole, lansoprazole, rabeprazole, dexiansoprazole, brefeldin A, golgicide, dynasore, pitstop, amodiaquine, EGA (4-bromobenzaldehyde N-(2,6-dimethylphenyl)semicarbazone) paroxetine, thioridazine, phenothiazine, promethazine, prochlorperzaine, trifluoperazine prochlorperzine, , quinine, Calcimycin, mefloquine, aprindine, disopyramide, flecainide, lidocaine, mexiletine, pentisomide, propafenone, cyproheptadine azatadine, loratadine, pizotifen, amitriptyline, propranolol, rupatadine, deptropine, amisulpride, nortriptyline, cyclobenzaprine, octriptyline, butriptyline, iprindole, trimipramine, flavoxate, cinnarizine chlomipramine, promazine, imipramine, carbamazepine, ay9944, clomipramine, clozapine, flecainide, ketoconazole, ofloxacin, perhexiline, sotalol, temoxiphen, zimelidine, toremifene, fluphenazine, trifluoperazine, pizotyline, CGS 12066B, prochlorperazine, ketotifen, lacidipine, sb 205607, lofepramine, mifepristone, clobenpropit, salmeterol, indacaterol, olodaterol, azelastine, epinastine, desloratadine, am-251, indatraline, nelfinavirhaloperidol, benproperine, M-paroxetine, carvedilol, calcipotriol, perphenazine, phenothiazine, chlorprothixene, desipramine, tetracaine, ifenprodil, U18666A, fluvoxamine maleate, prazosine, levomepromazine, prothipendyl, flupenthixol, zuclopentixol, clothiapine, bromperidol, droperidol, pipamperone, fluspirilene, pimozide, alectinib, brigatinib, crizotinib, ceritinib, bosutinib, imatinib, ponatinib, dasatinib, palbociclib, ribociclib, abemaciclib, sunitinib, afatinib, osimertinib, cobimetinib, 5-nonyloxytryptamine, alimemazine, dimenhydrinate, carteolol, betaxolol, levobunolol, timolol, proxymetacaine, brimonidine, apraclonidine, cyclopentolate oxybuprocaine, procyclidine, biperidene, trihexyphenidyl, vilanterol, and diphenhydramine, including salts and/or combinations thereof.

TABLE 2
Particularly interesting compounds in the context of the invention are disclosed in the below

| Compound | | MW | | |
|---|---|---|---|---|
| Name | Structure | (g/mol) | clogP | pKa1 |
| Desloratadine | 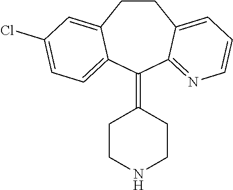 [SMILES: Clc1ccc2c(c1)CCc1cccnc1C2=C1CCNCC1] | 310.83 | 3.97 | 9.73 |
| Ketotifen | 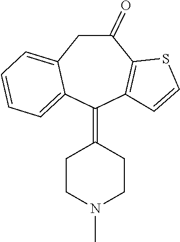 [SMILES: CN1CCC(=C2c3ccccc3CC(=O)c3sccc32)CC1] | 309.4 | 3.35 | 7.15 |
| Loperamide hydrochloride | 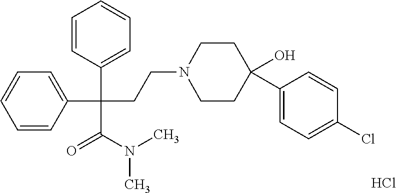 [SMILES: CN(C)C(=O)C(CCN1CCC(O)(c2ccc(Cl)cc2)CC1)(c1ccccc1)c1ccccc1 ; Cl] | 513.5 | 4.77 | 9.41 |
| Verapamil hydrochloride | 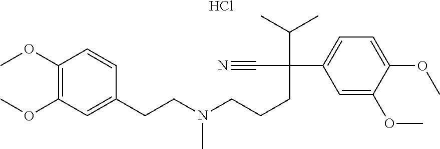 | 509.08 | 5.04 | 9.68 |
| Epinastine | 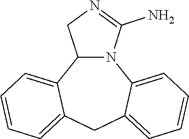 [SMILES: NC1=NCC2c3ccccc3Cc3ccccc3N12] | 249.31 | 3.13 | 9.31 |
| Nortriptyline hydrochloride | 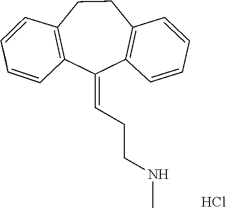 [SMILES: CNCC/C=C1\c2ccccc2CCc2cc3c(cc21)C3 ; Cl] | 299.84 | 4.51 | 10.1 |
| Amitriptyline hydrochloride | 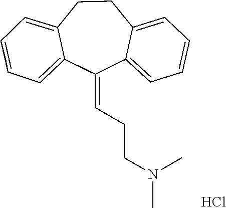 [SMILES: CN(C)CC/C=C1\c2ccccc2CCc2cc3c(cc21)C3 ; Cl] | 313.87 | 4.81 | 9.76 |
| Imipramine hydrochloride | 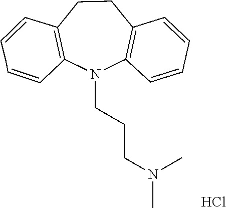 [SMILES: CN(C)CCCN1C2=C(C=CCC=C2)CCc2ccccc21 ; Cl] | 316.87 | 4.28 | 9.20 |
| Desipramine hydrochloride | 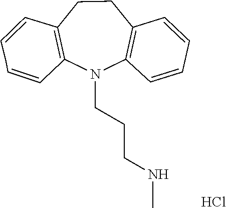 [SMILES: CNCCCN1C2=C(C=CCC=C2)CCc2ccccc21 ; Cl] | 302.85 | 3.90 | 10.02 |
| Fluoxetine | 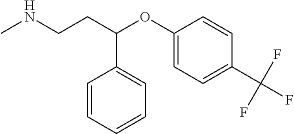 [SMILES: CNCCC(Oc1ccc(C(F)(F)F)cc1)c1ccccc1] | 309.33 | 4.17 | 9.78 |
| Fluvoxamine maleate | 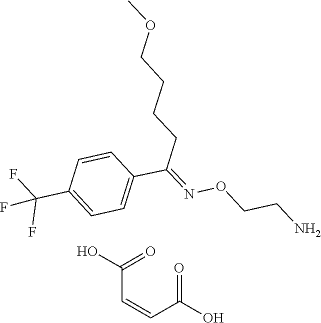 | 434.4 | 2.8 | 8.86 |

In one embodiment, the disclosed nanoparticles may include about 10 to about 90 mole percent, or about 5 to about 75 mole percent of a CAC, in particular a CAD as provided herein. In one embodiment, the disclosed nanoparticles include about 40 to about 60 mole percent, more specific about 50 mole percent of a CAC, in particular a CAD.

The term “nanocarrier” as used herein can be interpreted broadly and refers to a carrier being used as a transport module for another substance, such as a drug, in particular a macromolecular drug, more in particular a nucleic acid. Such carriers can be particles between about 5 nm to about 10 μm in size. The nanoparticles as referred to herein, e.g. as currently being studied for their use in drug delivery, range from sizes of diameter 10-1000 nm, in particular from about 10 to about 500 nm, or about 10 to about 300 nm, or about 10 to about 200 nm, or about 10 to about 150 nm. It should be appreciated that disclosed nanoparticles may be formed at a particular size, which may determine uptake pathways, circulation time, targeting, internalization, and/or clearance. Preferably, the size of the nanoparticle is such that it is capable of being taken up by a mammalian cell by endocytosis and is subsequently trafficked to endosomal organelles. Besides nanoparticles, also carriers with a size>1 μm can be internalized by phagocytic cell types (e.g. macrophages, dendritic cells) and are trafficked toward phagosomes. Because of their small size, nanoparticles can deliver drugs to otherwise inaccessible sites around the body. In particular, the present invention provides lipid-based particles, including lipid nanoparticles (LNPs) and lipoplexes (LPXs).

As known to the person skilled in the art, a lipid nanoparticle (LNP) typically comprises a selection of different lipid components, such as a cationic and/or ionizable lipid (also sometimes referred to as cationic ionizable lipid), a polymer conjugated lipid such as polyethylene glycol (PEG)-lipids and a (neutral) helper lipid such as a phospholipid. LNPs are often formulated via microfluidic mixing approaches. Optionally, the nanoparticle further comprises a steroid or sterol such as cholesterol or an analogue thereof. In general, a lipoplex (also referred to as cationic liposome complexed with an anionic nucleic acid) comprises or consists essentially of at least one nucleic acid molecule, and at least one (cationic) lipid.

In one embodiment, the invention provides a lipid-based nanoparticle comprising, consisting essentially of or consisting of at least one cationic amphiphilic compound (CAC) as provided herein, at least one nucleic acid molecule, and at least one lipid. In a particular embodiment, the lipid (also referred to as the lipid component) is a phospholipid, sphingolipid (such as sphingomyelin), lysolipid, mixed acyl lipid, ether lipid, ester lipid, oxidized lipid, cholesterol lipid, neutral lipid, zwitterionic lipid, charged lipid, sterol, sterol analogue, sterol-modified lipid, natural lipid, glycosylated lipid, pH-sensitive lipid, isoprenoids, bacterial lipid, plant lipid, bioactive lipid, lipid adjuvants, coenzyme A modified lipid, photo switchable lipids, click lipids, bile lipid, headgroup-modified lipid, fatty acid modified lipids, inverted headgroup lipid, polymer-conjugated lipid, polymerizable lipid, stabilizing lipid, or any combination thereof. Suitable lipids are generally known in the art.

In one embodiment, the lipid is a phospholipid. In the context of the present invention, the term “phospholipid” is meant to be a lipid molecule consisting of two hydrophobic fatty acid “tails” and a hydrophilic “head” consisting of a phosphate group. The two components are most often joined together by a glycerol molecule, hence, the phospholipid of the present invention is preferably a glycerol-phospholipid. Furthermore, the phosphate group is often modified with simple organic molecules such as choline (i.e. rendering a phosphocholine) or ethanolamine (i.e. rendering a phosphoethanolamine).

Suitable phospholipids can be selected from the group consisting of: 1,2-Dioleoyl-sn-glycero-3-phosphoethanolamine (DOPE), 1,2-Dioleoyl-sn-glycero-3-phosphocholine (DOPC), 1,2-distearoyl-sn-glycero-3-phosphocholine (DSPC), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-phosphatidylethanolamine (DSPE), 1,2-diundecanoyl-sn-glycero-phosphocholine (DUPC), 1-palmitoyl-2-oleoyl-sn-glycero-3-phosphocholine (POPC), stearoyloleoylphosphatidylcholine (SOPC), 1-stearoyl-2-oleoyl-phosphatidyl ethanol amine (SOPE), L-a-phosphatidylcholine (Egg, Chicken), L-a-phosphatidylcholine (Soy), L-a-phosphatidylcholine (Brain, Porcine) 1,2-dioleoyl-sn-glycero-3-phosphatidylglycerol (DOPG), 1,2-dioleoyl-sn-glycero-3-phosphatidic acid (DOPA), 1,2-dipalmitoyl-sn-glycero-3-phosphoethanolamine (DPPE), 1,2-dipalmitoyl-sn-glycero-3-phosphatidylglycerol (DPPG), 1,2-dipalmitoyl-sn-glycero-3-phosphatidic acid (DPPA), 1-oleoyl-sn-glycero-2,3-cyclic-phosphate (18:1 cyclic LPA), L-a-phosphatidic acid (Soy), L-a-phosphatidic acid (Egg, Chicken), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1-O-octadecyl-2-O-methyl-sn-glycero-3-phosphocholine (Edelfosine), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1.2-distearoyl-sn-glycero-3-phosphoethanolamine, 1,2-dilinoleoyl-sn-glycero-3-phosphoethanolamine, 1,2-dilinolenoyl-sn-glycero-3-phospho-ethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt, 1,2-dierucoyl-sn-glycero-3-phosphocholine, 1,2-distearoyl-sn-glycero-3-phosphoinositol (18:0 PI), 1,2-dioleoyl-sn-glycero-3-phospho-(1′-myo-inositol) (18:1 PI), 1,2-dipalmitoyl-sn-glycero-3-phospho-(1′-myo-inositol) (16:0 PI), 1-palmitoyl-2-oleoyl-sn-glycero-3-phosphoinositol (16:0-18:1 PI), 1,2-dioleoyl-sn-glycero-3-phospho-(1′-myo-inositol-3′-phosphate) (18:1 PI(3)P), 1,2-dioleoyl-sn-glycero-3-phospho-(1′-myo-inositol-3′,4′-bisphosphate) (18:1 PI(3,4)P2), 1,2-dioleoyl-sn-glycero-3-phospho-(1′-myo-inositol-3′,4′,5′-trisphosphate) (18:1 PI(3,4,5)P3), 1,2-dipalmitoyl-sn-glycero-3-phospho-L-serine (16:0 PS), 1,2-distearoyl-sn-glycero-3-phospho-L-serine (18:0 PS), 1,2-dioleoyl-sn-glycero-3-phospho-L-serine (DOPS), 1-palmitoyl-2-oleoyl-sn-glycero-3-phospho-L-serine (POPS), 1-oleoyl-2-hydroxy-sn-glycero-3-phospho-L-serine (18:1 lyso PS), 1′,3′-bis[1,2-dipalmitoyl-sn-glycero-3-phospho]-glycerol (16:0 cardiolipin), 1′,3′-bis[1,2-dioleoyl-sn-glycero-3-phospho]-glycerol (18:1 cardiolipin), 1′,3′-bis[1-palmitoyl-2-oleoyl-sn-glycero-3-phospho]-glycerol (16:0-18:1 cardiolipin), heart cardiolipin (bovine), bis(monooleoylglycero)phosphate (S,R Isomer) (18:1 BMP (S,R), sn-[2,3-dioleoyl]-glycerol-1-phospho-sn-1′-[2′,3′-dioleoyl]-glycerol (18:1 BDP (S,S), oxidized 1-palmitoyl-2-arachidonoyl-sn-glycero-3-phosphocholine (oxPAPC), 2-((2,3-bis(oleoyloxy)propyl)dimethylammonio)ethyl hydrogen phosphate (DOCP), 1-O-hexadecyl-2-oleoyl-sn-glycero-3-phosphocholine (C16-18:1 PC), 1-O-octadecyl-2-hydroxy-sn-glycero-3-phosphocholine (C18 lyso PAF), and derivatives, enantiomers, salts or mixtures thereof.

In a particular embodiment, the phospholipid is DOPE or DSPC, or a combination thereof.

In another embodiment, the nanoparticle of the invention, more specific the LNP, further comprises a steroid or a sterol, more preferably cholesterol. Incorporation of a steroid or a sterol in the LNP promotes (extracellular) stability of the particle. As defined herein, “sterols” are a subgroup of steroids consisting of steroid alcohols. A particularly preferred sterol is cholesterol or an analogue thereof, such as DC-Cholesterol, 3beta-[N—(N′,N′-dimethylaminoethane)-carbamoyl]cholesterol, 1,4-bis(3-N-oleylamino-propyl)piperazine or ICE, fucosterol, sitosterol, ergosterol, phytosterols, campesterol, stigmasterol, brassicasterol, tomatidine, ursolic acid, alpha-tocopherol, and diterpenoids or triterpene alkaloids.

In another embodiment, the lipid-based nanoparticle as envisaged herein may further include one or more PEGylated lipids. A PEGylated lipid is a lipid modified with polyethylene glycol. This may improve the stability, biodistribution and biocompatibility of the LNP through shielding surface charge, avoiding aggregation, extending circulation time, modulating interaction with proteins and opsonization, reducing off-target interactions, reducing phagocytic clearance and the like. A PEGylated lipid may be selected from the non-limiting group consisting of PEGylated phosphatidylethanolamines, PEGylated phosphatidic acids, PEGylated ceramides, PEGylated dialkylamines, PEGylated diacylglycerols, PEGylated dialkylglycerols, PEGylated sterols, and mixtures thereof. PEGylated lipids can be additionally functionalized, such as 1,2-distearoyl-sn-glycero-3-phosphoethanolamine-N-[amino(polyethylene glycol)-2000](DSPE-PEG(2000) amine), 1,2-distearoyl-sn-glycero-3-phosphoethanolamine-N-[folate(polyethylene glycol)-2000](DSPE-PEG(2000) folate) or 1,2-distearoyl-sn-glycero-3-phosphoethanolamine-N-[maleimide(polyethylene glycol)-2000](DSPE-PEG(2000) maleimide). Preferably, the PEGylated lipid is selected from the group consisting of DMG-PEG, DSPE-PEG, DSG-PEG, or 2-[(polyethylene glycol)-2000]-N,N-ditetradecylacetamide, wherein the molecular weight of PEG ranges from 1-10 kDa. In certain embodiments, the PEGylated lipid is a PEG-OH lipid. A “PEG-OH lipid”, also referred to herein as “hydroxy-PEGylated lipid”, is a PEGylated lipid having one or more hydroxyl (—OH) groups on the lipid and/or on the PEG chain. In certain embodiments, a PEG-OH or hydroxy-PEGylated lipid comprises a hydroxyl group at the terminus of the PEG chain. In a particular embodiment, the PEGylated lipid is DMG-PEG.

In one embodiment, the disclosed nanoparticles may include about 0 to about 50 mole percent, about 1 to about 45 mole percent, about 1.5 to about 40 mole percent, about 2 to about 30 mole percent, about 4 to about 20 mole percent of a PEGylated lipid.

In one aspect, the LNPs of the present invention are typically composed of a combination of a CAC, a (helper) lipid such as a phospholipid, a sterol, a PEGylated lipid and optionally a cationic and/or ionizable lipid.

Usually the cationic or ionizable lipid is the dominant component as it enables both electrostatic complexation of the oppositely charged RNA as well as cellular delivery by facilitating cellular uptake and endosomal escape. To date, many cationic/ionizable lipid materials have been synthetized for LNP production. However, the cytosolic delivery efficiency often remains poor.

Moreover, concerns remain regarding the safety and immunogenicity of these synthetic cationic lipid-like materials, e.g. when repeated administration is required for chronic treatment. As such, to expedite clinical translation of this highly promising class of therapeutics, lipid-based nucleic acid formulations are needed that merge efficient cellular delivery with acceptable toxicity.

It has been shown in the present invention that the CAC as provided herein can be used to partly (about 5 mole percent to about 90 mole percent, about 25 mole percent to about 75 mole percent or about 50 mole percent) or fully (i.e. 100 mole percent) replace the cationic or ionizable lipids in lipid-based nanoparticles. Hence, this invention describes the repurposing of CACs as both structural and functional components of lipid-based nanoparticles, fully replacing (synthetic) cationic/ionizable lipids (also referred to herein as CADosomes) or supplementing (synthetic) cationic/ionizable lipids (also referred to herein as CAD-LNPs; wherein part of the other lipids (such as sterol e.g. cholesterol component) typically used, is replaced by a CAC), showing functional delivery of RNA, both small RNA and mRNA, into target cells. Hence in a specific embodiment, the invention provides a nanoparticle comprising or consisting essentially of a CAC, a nucleic acid, a PEG lipid and a helper lipid, and wherein said nanoparticle does not comprise a cationic/ionizable lipid.

On the other hand, by selectively replacing (part of) the cholesterol fraction by a CAC, the delivery efficiency of both siRNA and mRNA could even be increased. Accordingly, the nanoparticle of the invention comprises or consist essentially of a CAC, a nucleic acid and a cationic/ionizable lipid, and wherein said nanoparticle does not comprise or comprises a relatively low amount of a sterol (e.g. cholesterol or analogues), such as about 5-15 or 5-20 mol % of a sterol, such as about 5, 6, 7, 8, 9, 10, 11, 12, 13, 14, 15, 16, 17, 18, 19 or 20 mol % of a sterol.

As mentioned herein before, CACs can be added to existing LNPs to supplement cationic/ionizable lipids in the formulation (CAD-LNPs). As such, the nanoparticle provided herein may further comprise (a reduced amount of) a cationic or ionizable lipid. “Ionizable lipids”, when formulated in LNPs, typically have a pKa<7.4 and have a neutral to mildly cationic charge under physiological pH conditions.

In the context of the present invention the term “ionizable” (or alternatively cationic) in the context of a compound or lipid means the presence of any uncharged group in said compound or lipid which is capable of ionizing by acquiring an ion (usually an H+ion) and thus itself becoming positively charged. Alternatively, any uncharged group in said compound or lipid may dissociate and yield an ion (usually an H+ion) and thus becoming negatively charged. In the context of the present invention any type of cationic or ionizable lipid can suitably be used. Examples of cationic or “ionizable” cationic lipids are well known to the skilled person and include 4-hydroxybutyl)azanediyl)bis(hexane-6,I-diyl)bis(2-hexyldecanoate; 1,2-dioleoyl-3-trimethyl-ammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-I-(cis,cis-9,12-oc-tadecadienoxy)propane (CLinDMA), 2-[5′-(cholest-5-en-3-beta-oxy)-3′-oxapentoxy)-3-dimethyl-I-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), 1,1′-((2-(4-(2-((2-(bis(2-hydroxydodecyl)amino)ethyl)(2-hydroxydodecyl)amino)ethyl)piperazin-1-yl)ethyl)azanediyl) bis(dodecan-2-ol) (C12-200), heptatriaconta-6,9,28,31-tetraen-19-yl-4-(dimethylamino) butanoate (DLin-MC3-DMA), [(4-hydroxybutyl)azanediyl]di(hexane-6,1-diyl) bis(2-hexyldecanoate) (ALC-0315), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-!-propanaminium bromide (GAP-DLRIE), (±)-N-(3-aminopropyl)-N,N-dimethyl-2, 3-bis(tetradecyloxy)-1-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy)propan-I-aminium (DOBAQ), 2-{8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), NI-[2-((IS)-I-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy)propan-1-ammonium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid 02-200); or heptadecan-9-yl 8-((2-hydroxyethyl) (6-oxo-6-(undecyloxy) hexyl) amino) octanoate (SM-102). In one embodiment, the ionizable lipid is DLin-MC3-DMA (MC3).

In a specific embodiment, the disclosed LNPs may include about 1 to about 90 mole percent, about 1 to about 75 mole percent, or about 5 to about 70 mole percent of a cationic and/or ionizable lipid.

The nanoparticle as provided herein may be customized in terms of size, shape, surface charge and attachment of any targeting moieties such as e.g. antibodies, antibody fragments, peptides, folate, transferrin, apolipoproteins, carbohydrates (such as mannose, galactose or GaINAc), haloperidol, anisamide, and cardiac glycosides or the like.

### Active Agent

The nanoparticles of the present invention are suitable for use with any (therapeutic) agent. The agent may be encapsulated by the nanoparticle and/or it may be attached to a surface or surfaces thereof to form a conjugate. In some cases, the encapsulation of the therapeutic agent is advantageous, as higher concentrations of a drug can be encapsulated and the drug is protected from the interaction with external components (e.g. degradative enzymes). Suitable methods for encapsulating agents inside nanoparticles are known to the skilled person and comprise electrostatic complexation, covalent coupling, hydrophobic interactions, passive loading, remote loading, salting-out, nanoprecipitation, emulsion-diffusion, solvent-evaporation, spray drying and emulsion polymerization. Typically such methods may be adapted depending upon the materials used to make the nanoparticles and the chosen agent, which adaptation will be within the remit of the skilled person. As used herein, the “active agent” may be a protein, peptide, lipid, (poly)saccharide, chemical compound, imaging agent, genetic material (i.e. a nucleic acid) or any other (biologically) active molecule.

In a particular embodiment, the active agent is genetic material, i.e. a nucleic acid, including but not limited to one or more of DNA, RNA, hybrids thereof, plasmid DNA, transposons, messenger RNA, single guide RNA (sgRNA), RNAi-inducing agents, antisense RNAs, antisense DNAs, ribozymes, cyclic nucleic acids, catalytic DNA, RNAs that induce triple helix formation, aptamers, and vectors. The nucleic acids may be single strand (sense or antisense), or double strand. The nucleic acids may be unmodified or modified. It has been demonstrated in the present invention that the CAC is employed both as an essential structural and functional component as it allows the formation of cationic vesicles in combination with one or more lipids owing to its charge and amphiphilicity, enables electrostatic complexation of e.g. RNA because of its positive charge, and contributes to cytosolic delivery of nucleic acids.

In particular, the nucleic acid is RNA. In one embodiment, the nanoparticles may include small nucleic acid molecules such as such as short interfering RNA (siRNA), double stranded RNA (dsRNA), micro-RNA (miRNA), small nucleolar RNA (sno-RNA), transfer RNA (tRNA), Piwi-interacting RNA (piRNA), single guide RNA (sgRNA) or short hairpin RNA (shRNA). In other embodiments, messenger RNAs (mRNAs) or long noncoding RNA (lncRNA) may be incorporated into the nanoparticle.

Particularly envisaged in the present invention are complexes of a nanoparticle and mRNA. A mRNA may be a naturally or non-naturally occurring mRNA. A mRNA may include one or more modified nucleobases, nucleosides, or nucleotides. A nucleobase of a mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction. The mRNA may include a 5′ untranslated region, a 3′ untranslated region, and/or a coding or translating sequence. Optionally, the mRNA includes one or more of a stem loop, a chain terminating nucleoside, a polyA sequence, a polyadenylation signal, and/or a 5′ cap structure. The mRNA may also be a self-amplifying mRNA.

Further envisaged are complexes of a nanoparticle and a small non-coding RNA or antisense oligonucleotide (typically up to 30 nucleotides e.g. 13-25 nucleotides). RNA interference (RNAi) represents a powerful gene silencing mechanism wherein ˜21 nt RNA duplexes, i.e. siRNAs, function as the effector molecules for sequence-specific mRNA cleavage, thereby inducing sequence-specific gene-silencing on the post-transcriptional level. Since synthetic siRNAs have been shown to activate the RNAi pathway and since they can be designed to target nearly any human gene, RNAi has become the method of choice to suppress gene expression for therapeutic purposes. Also siRNAs may be envisaged with canonical or non-canonical nucleobases, nucleosides or nucleotides.

The amount of nucleic acid, in particular RNA, in a nanoparticle may depend on the size, sequence, and other characteristics of the RNA and/or on the size, composition, desired target, and other characteristics of the nanoparticle composition. The relative amounts of nucleic acid and other elements (e.g., CAC, lipids) may also vary.

In one embodiment, the wt/wt ratio of the lipid components to an mRNA in the nanoparticle composition may be from about 1:1 to about 100:1. For example, the wt/wt ratio of the lipid components to an mRNA may be from about 2:1 to about 75:1, from about 5:1 to about 50:1, from about 10:1 to about 30:1. In a preferred embodiment, the wt/wt ratio of the lipid components to the mRNA is about 10:1. In some embodiments, the mRNA, lipids, and amounts thereof maybe selected to provide a specific N:P ratio. The N:P ratio of the composition refers to the molar ratio of nitrogen atoms (e.g. present in a CAC or cationic lipid) to the number of phosphate groups in an mRNA. The mRNA, lipids and amounts thereof may be selected to provide an N:P ratio from about 1:2 to about 30:1, such as 1:2, 1:1, 2:1, 5:1, 6:1, 7:1, 8:1, 9:1, 10:1, 11:1, 12:1, 13:1, 14:1, 15:1, 20:1, 25:1 and 30:1, and in particular from about 3:1 to 15:1.

The amount of mRNA complexed by a nanoparticle composition may, for example, be measured using gel electrophoresis, fluorescence spectroscopy (e.g., fluorescence correlation spectroscopy (Zhang et al., 2018)), UV-VIS spectroscopy or the ribogreen assay. The latter assay (e.g. in combination with a detergent such as Triton-X-100) can additionally be used to quantify the encapsulated mRNA dose.

Ratios can be evaluated via dynamic light scattering and electrophoretic mobility measurements.

In a further embodiment, the nanoparticles may include an imaging agent for example fluorine compounds, such as perfluorocarbon (PFCs), and fluorescent labels, such as fluorescent dyes, well known to the skilled person. Examples of suitable fluorescent labels include fluorescein (such as fluoresceinamine or fluorescein isothiocyanate (FITC)), rhodamine, Alexa Fluor® dyes, DyLight® Fluor dyes, ATTO dyes, boron-dipyrromethene (BODIPY) dyes and such like.

The presence of an imaging agent permits the nanoparticle to be tracked in cells in vitro and/or in vivo. The imaging agent may be included in the nanoparticle by any suitable means including encapsulation, covalent conjugation, physical immobilisation (for example, by electrostatic attraction, hydrophobic interaction and such like), layer-by-layer (LbL) adsorption and so on. The particular method used will depend upon the particular imaging agent and the nanoparticles selected, and such methodology would be within the remit of a skilled person.

### Use

The present invention furthermore encompasses a nanoparticle comprising an active agent such as a nucleic acid, at least one cationic amphiphilic compound having a c log P value of less than 10, and at least one helper compound, in particular a helper lipid, for use as a medicament. The combination of the present invention is particularly useful for medical applications such as human as well as veterinary therapeutic, diagnostic or theranostic applications. Despite numerous efforts, endosomal escape remains an inefficient process up to date and consequently lysosomal entrapment is regarded as a non-functional dead end for nucleic acid based nanomedicines. Surprisingly, the nanoparticles of the invention outperformed both commercially available DOTAP:DOPE lipoplexes, as well as state-of-the-art ionizable LNPs with the DLin-MC3-DMA lipid (prepared via microfluidic mixing) in a primary PBCEC model.

The method as described herein induces release of the accumulated active agent in the cytosol by use of a specific CAC. Intracellular events can be more effectively affected and regulated upon intracellular delivery of different biologically active agents using said nanoparticles. These active agents may modify or normalize the cellular function or may eliminate unwanted cells when needed. The changing of the cellular functionality may involve a change in a physico-chemical property of the cell, a change in proliferative property of the cell, a change in surviving ability of the cell, a change in secretory capacity of the cell, a change in migration property of the cell or a change in morphological phenotypical property of the cell. Furthermore, and as an advantage over conventional small molecule inhibitors and monoclonal antibodies, RNA drugs can target virtually any human gene, offering a broad spectrum of biomedical applications.

Examples are intracellular delivery of tumor antigen-encoding mRNA in dendritic cells or siRNA-induced downregulation of immunosuppressive pathways in cytotoxic T cells for cancer immunotherapy. Furthermore, the nanoparticles of the invention, in particular the CADosomes, showed to be a suitable carrier for RNA delivery in human ex vivo explant models or for in vivo application to cells that are hard to transfect, such as HUVECs, stem cells, immune cells, and neuron cells, in particular corneal cells. Hence, many clinical applications, both via local and systemic administration, can be envisaged.

In a particular embodiment, the nanoparticles as provided herein are useful for the prophylaxis and/or treatment of various diseases such as cancer, infectious disease, autoimmune disease, genetic disorders, etc.

In a further embodiment, therapies comprising the use of the nanoparticle and methods of treatment using such therapies are provided. In one embodiment, a therapy comprises administration of a nanoparticle as defined herein.

Next to in vivo application, also several in vitro/ex vivo applications can be considered. The nanoparticles provided herein can be used as in vitro transfection reagents for biomedical research, cell biology and/or biomanufacturing, for controlling gene expression in cells of interest. Ex vivo, the nanoparticles can be applied as transfection reagent in cell-based therapies, where the properties of cells obtained from a patient are modified ex vivo prior to re-administration to obtain a therapeutic effect. Examples are the ex vivo engineering of immune cells (T cells, NK cells, dendritic cells) or gene correction in hematopoietic stem cells.

In one embodiment, the invention provides a method for delivering an agent, in particular a nucleic acid, into the cytosol of a cell by in vitro, ex vivo or in vivo application whereby the nanoparticle provided herein is administered to a cell or subject.

The methods of intracellular delivery as herein provided can be applied in any context wherein delivery of materials across the cell membrane is required, such as but not limited to drug screening, drug delivery, imaging, cell labeling, cell engineering, manufacturing of cell therapies, in particular adoptive T cell therapies, and the like. It is accordingly an object of the present invention to provide the use of the methods as herein provided in drug screening, drug delivery, imaging, cell labeling, cell engineering, manufacturing of cell therapies, adoptive T cell therapies, and the like. A variety of cell types can be transfected, including hard-to-transfect primary corneal epithelial cells and primary human T cells. The methods can be applied to single cells, cell cultures, isolated cells, cells in suspension or grown on substrates such as culture dish, both in in vivo, in vitro and ex vivo applications, and typically include contacting the cells with the nanoparticles provided herein.

The nanoparticle of the invention may be used in a monotherapy for treating, ameliorating, reducing the risk of or preventing a disease. Alternatively, the nanoparticle may be used as an adjunct to, or in combination with, known therapies which may be used for treating, ameliorating, reducing the risk of or preventing a disease.

The present invention furthermore encompasses a nanoparticle comprising an active agent such as a nucleic acid, at least one cationic amphiphilic compound as provided herein and at least one helper compound, in particular a helper lipid, for use in combination therapy. From a therapeutic perspective, the intrinsic pharmacological effect of the CAD (e.g. chemotherapeutic, anti-inflammatory agent, anti-viral agent etc.) could synergistically contribute to the envisioned therapeutic response.

### Production of the Nanoparticles

Different preparation protocols for LNPs are known to the skilled person. One particular method is rapid (microfluidic) mixing of an ethanolic solution of CACs and lipids with an aqueous solution of nucleic acid drug, to maximize drug loading, encapsulation efficiency and stability. Other methods have also been described and are readily available to the skilled person.

In one embodiment, the present invention provides a method for preparing a nanoparticle comprising one or more of the following
Microfluidic mixing
Spontaneous particle formation by solvent (ethanol) dilution method
Nanoprecipitation technique
Solvent evaporation method
Emulsification-based methods
Phase separation methods
Crossflow injection technique
T-junction mixing
Micro hydrodynamic focusing
Microfluidic droplets
Pulsed jet flow microfluidics
Thin-film hydration
Supercritical reverse phase evaporation
One-step spray drying method
Membrane contactor method

Alternatively, lipid-based nanoparticles can also be prepared via the classic two-step approach, in which first (cationic) lipid vesicles/liposomes are formed (e.g. via lipid film hydration or ethanol dilution) for subsequent complexation of nucleic acid.

In another embodiment, the present invention provides a method for preparing a nanoparticle comprising one or more of the following:
Thin-film hydration+extrusion
Thin-film hydration+sonication
Preformed vesicle method
Ethanol-injection or ethanol drop method
Injection of organic solvent with dissolved phospholipids into an aqueous phase
Encapsulation of nucleic acid in ethanol-destabilized liposomes
Detergent dialysis method
Reverse-phase evaporation method
Electro-formation method
Coalescence of small vesicles
Solvent spherule method
Hydration of proliposomes
Size reduction methods of MLVs and GUVs
Supercritical fluids (SCFs) method
Modified Electroformation methods
Freeze drying of double emulsions
Hydration of phospholipids deposited on nanostructured material
Formation by curvature tuning
Biomimetic reaction for vesicle self-assembly

### Pharmaceutical Compositions

The invention further provides pharmaceutical compositions or delivery systems comprising the lipid-based nanoparticle comprising at least one CAC as provided herein and a pharmaceutically acceptable excipient, carrier and/or diluent, and optionally at least one adjuvant as described herein. The optional ingredients will depend on the application and will be determined by the skilled person. The invention provides first and further medical uses of the nanoparticle (or the composition comprising it) and the CACs as provided herein. More particular, the present invention provides the CAC-nanoparticle of the invention for use in the intracellular delivery of an agent, especially a membrane-impermeable agent or a hydrophobic agent or drug, in particular a nucleic acid.

A “pharmaceutically acceptable excipient” as referred to herein, is any known compound or combination of known compounds that are known to those skilled in the art to be useful in formulating pharmaceutical compositions. In one embodiment, the pharmaceutically acceptable excipient may be a solid. A solid pharmaceutically acceptable excipient may include one or more substances which may also act as stabilizers, flavouring agents, lubricants, solubilisers, suspending agents, fillers, glidants, compression aids, inert binders, sweeteners, preservatives, dyes, coatings, or tablet-disintegrating agents. The excipient may also be an encapsulating material. In powders, the excipient is a finely divided solid that is in admixture with the finely divided active agents according to the invention. Suitable solid excipients include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like. In addition, the pharmaceutical excipient may be a liquid, and the pharmaceutical composition may be in the form of a solution. Liquid excipients are used in preparing solutions, suspensions, emulsions, ionic liquids, syrups, elixirs and pressurized compositions. The nanoparticle, active agent and/or adjuvant according to the invention may be dissolved or suspended in a pharmaceutically acceptable liquid excipient such as water, an organic solvent, a mixture of both or pharmaceutically acceptable oils or fats. The liquid excipient can contain other suitable pharmaceutical additives such as stabilizers, solubilisers, emulsifiers, buffers, preservatives, sweeteners, flavouring agents, suspending agents, thickening agents, colours, viscosity regulators, stabilisers or osmo-regulators. Suitable examples of liquid excipients for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the excipient can also be an oily ester such as ethyl oleate and isopropyl myristate. Sterile liquid excipients are useful in sterile liquid form compositions for parenteral administration. Liquid pharmaceutical compositions, which are sterile solutions or suspensions, can be utilised by, for example, subcutaneous, intrathecal, epidural, intraperitoneal, intravenous and intramuscular injection.

Compositions comprising the nanoparticles may be administered in a number of ways, e.g. by oral administration, by inhalation (e.g. intranasally or orally), by injection (into the blood stream or directly into a site requiring treatment), as topical use, or incorporated within a slow- or delayed-release device. In a particular embodiment, the administration is by intramuscularly, intravenous (bolus or infusion), subcutaneous (bolus or infusion), or intradermal (bolus or infusion) injection. In another embodiment, the administration or use is topical, in particular for the delivery of an active agent to the skin epithelium, and more particularly for topical skin applications for treatment of skin disorders and maladies. Besides topical applications to skin, the methods of the present invention are equally useful in the delivery of the nanoparticles, including the active agents therein, to the cornea. In said embodiment of the corneal application, the nanoparticles are provided as an ophthalmic solution, optionally comprising as further ingredients buffer, tonicity agent, solubilizer, surfactant, stabilizer, preservative, pH adjuster, and the like.

The frequency of administration will be influenced by the half-life of the active agents within the subject being treated. Optimal dosages to be administered may be determined by those skilled in the art, and will vary with the particular adjuvants and nanoparticles or cells in use, the stability of the pharmaceutical composition, the mode of administration, and the advancement of the disease. Additional factors depending on the particular subject being treated will result in a need to adjust dosages, including subject age, weight, gender, diet and time of administration. The nanoparticles may be administered before, during or after onset of the disease, disorder or condition to be treated. Daily doses may be given as a single administration of the combination as described herein (e.g. a single daily injection). Alternatively, administration can be twice or more times during a day.

A “subject”, as used herein, may be a vertebrate, mammal or domestic animal. Hence, nanoparticles, medicaments, or compositions according to the invention may be used to treat animals and humans, including any mammal, for example livestock (e.g. a horse), pets, and may be used in veterinary or human applications. Most preferably, the subject is a human being.

All of the features described herein (including any accompanying claims, abstract and drawings), and/or all of the steps of any method or process so disclosed, may be combined with any of the above aspects in any combination, except combinations where at least some of such features and/or steps are mutually exclusive.

### Examples

### Materials and Methods

Messenger RNA constructs. The 996 nucleotide long, CleanCap® Enhanced Green Fluorescent Protein Messenger RNA (eGFP-mRNA), modified with 5-methoxyuridine (5-moU), a Cyanine-5 labeled CleanCap® Enhanced Green Fluorescent Protein Messenger RNA (Cy5-mRNA), modified with 5-methoxyuridine (5-moU) and a nucleoside-modified (5meC, ψ) mRNA encoding firefly luciferase (fLuc-mRNA), were purchased from TriLink (San Diego, CA). Cre-recombinase mRNA (Cre-mRNA) was synthesized by the lab of Prof. Pieter Vader (Laboratory of Clinical Chemistry and Hematology, University Medical Center Utrecht, Utrecht, The Netherlands). The mRNA stocks were dissolved in nuclease-free water (Ambion©-Life Technologies, Ghent, Belgium) and stored in small aliquots at −80° C. at a concentration of 1 μg/μL. The mRNA stock concentration was determined from absorption measurements at 260 nm with a NanoDrop 2000c UV-Vis spectrophotometer (Thermo Fisher Scientific, Rockford, USA).

Preparation of CAD-DOPE nanoparticles. The cationic amphiphilic drugs (CADs) nortriptyline hydrochloride (NT), amitriptyline hydrochloride (AMI), desipramine hydrochloride (DSI), imipramine hydrochloride (IMI), ketotifen (KET), loperamide hydrochloride (LOP) and verapamil hydrochloride (VER) were purchased from Sigma-Aldrich (Overijse, Belgium) and desloratadine (DES) and epinastine hydrochloride (EP) from Cayman Chemical (Michigan, USA). The lipids DOTAP (1,2-dioleoyl-3-trimethylammonium-propane) and DOPE (1,2-dioleoyl-sn-glycero-3-phosphoethanolamine) were purchased from Avanti Polar Lipids (Alabaster, USA). Two literature-based methods were used to produce CAD-DOPE vesicles (MacLachlan et al. 2007; Buyens et al., 2012; Meisel et al., 2016).

For the ethanol dilution (ED) method, CADs (10 mg/ml) and DOPE (25 mg/ml) were separately dissolved in absolute ethanol. CAD-DOPE vesicles (50:50 mol ratio) were prepared by transferring the appropriate amount of CADs and DOPE into a round-bottom flask, followed by dropwise addition of 4-(2-hydroxyethyl)-1-piperazineethanesulfonic acid (HEPES) buffer (pH 7.4, 20 mM) and tip-sonication (6×10 sec, amplitude 10%; 10 sec on/15 sec off; Branson Digital Sonifier*, Danbury, USA). The final CAD-DOPE concentration after dilution in HEPES buffer (pH 7.4, 20 mM) was 3.56 mM. For the lipid film hydration method CAD (10 mg/ml) and DOPE (25 mg/ml) were dissolved in chloroform (50:50 mol ratio) and transferred to a brown glass vial, followed by chloroform evaporation under nitrogen flow. Next, the CAD-DOPE lipid film was rehydrated with HEPES buffer (pH 7.4, 20 mM), followed by tip-sonication and further dilution in HEPES buffer (pH 7.4, 20 mM) to obtain a final CAD-DOPE concentration of 3.56 mM. The classically used cationic DOTAP-DOPE liposomes (50:50 mol ratio) were likewise prepared via lipid film hydration, as previously reported in literature and diluted in HEPES buffer (pH 7.4, 20 mM) to obtain a final lipid concentration of 3.9 mM. Hydrodynamic diameter, polydispersity index (PDI) and zeta-potential of all formulations were determined via dynamic light scattering (DLS) (Zetasizer Nano, Malvern Instruments, Worcestershire, United Kingdom).

Physicochemical analysis of mRNA CADosome/lipoplexes. To obtain stable mRNA CAD-DOPE mRNA complexes (further denoted as CADosomes) and DOTAP-DOPE mRNA lipoplexes, pre-formed CAD-DOPE vesicles (without mRNA) and DOTAP-DOPE liposomes (without mRNA) were complexed with equal volumes of mRNA diluted in nuclease-free water (Ambion©-Life Technologies, Ghent, Belgium) for 10 min at room temperature, reaching the desired nitrogen-to-phosphate (N/P) ratio. Hydrodynamic diameter, polydispersity index (PDI) and zeta-potential of mRNA CADosomes and DOTAP-DOPE lipoplexes were determined via dynamic light scattering (DLS) (Zetasizer Nano, Malvern Instruments, Worcestershire, United Kingdom). Transmission electron microscopy (TEM) images of mRNA CADosomes were recorded at the VIB-UGent Biolmaging Core facility with a transmission electron microscope JEM1400plus operating at 80 kV (JEOL, Tokyo, Japan). The samples were prepared by depositing a drop (15 μL) of mRNA CADosomes on a formvar/C-coated hexagonal copper grid (EMS G200H-Cu), followed by five repeated washing steps in double-distilled water and a final staining with in uranyl-acetate. The samples were allowed to dry at room temperature. The complexation efficiency of mRNA CADosomes was measured via Fluorescence Correlation Spectroscopy (FCS) to quantify the complexation of fluorescently labeled RNA nanocarriers. Briefly, this microscopy-based technique monitors fluorescence intensity fluctuations of fluorescent molecules diffusing in and out of the fixed excitation volume of a confocal microscope. Fluorescence time traces (60s) were recorded by focusing a 640 nm laser line through a water immersion objective lens (60× Plan Apo VC, NA 1.2, Nikon, Japan) of a confocal microscope (Nikon C₂, Japan) at about 50 μm above the bottom of a glass-bottom 96-well plate (Grainer Bio-one, Frickenhausen, Germany), which contained 50 μL Cy5-mRNA CADosomes, naked Cy5-mRNA or blank HEPES buffer (pH 7.4, 20 mM) respectively. The fluorescence signal was recorded by a photon counting instrument (Pico-Harp 300, PicoQuant, Berlin, Germany) equipped with MATLAB software (MathWorks, USA).

Cell lines and culture conditions. The human cervical epithelial adenocarcinoma HeLa cell line was obtained from American Type Culture Collection (ATCC, Manassas, USA). A DsRed+HeLa reporter cell line, stably expressing the fluorescent Cre-recombinase reporter plasmid (pLV-CMV-LoxP-DsRed-LoxP-eGFP), was used. HeLa cells were cultured in Dulbecco's Modified Eagle Medium: Nutrient F-12 (DMEM/F-12) (Gibco©-Life Technologies, Grand Island, NY, USA), supplemented with 10% fetal bovine serum (FBS, Hyclone™, GE Healthcare, Machelen, Belgium), 2 mM L-Glutamine and 100 U/mL penicillin/streptomycin (hereafter collectively called ‘complete cell culture medium’ or CCM). The development of a Human Embryonic Kidney cell line (HEK293T), stably expressing the 5-HT2AR (Serotonin 2A Receptor) and the cytosolic protein μ-arrestin 2 (βarr2) in the NanoBiT© system. To obtain primary bovine corneal epithelial cells (PBCECs), freshly excised bovine eyes were collected at a local slaughterhouse (Flanders Meat Group, Zele, Belgium) and were transferred within 30 min in cold CO₂ independent medium. The eyes were cleaned of excess tissue and then disinfected by dipping into a 5% ethanol solution. A trephine blade was used to collect 10 mm diameter corneal buttons. The corneal buttons were rinsed with DMEM (Gibco©-Life Technologies, Grand Island, NY, USA) containing antibiotics and divided in 4 equal parts using a scalpel, rinsed again with DMEM and placed in a 15 mg/ml Dispase II solution at 37° C. for 15-30 min. Hereafter the tissues were rinsed with PBS and the epithelial layer was separated from the corneal stroma using stainless tweezers. The obtained epithelial sheets are placed in separate wells with fresh DMEM containing antibiotics, 4.5 g/L D-glucose and 10% FBS. Once 80% confluency was reached in the wells, the epithelial tissue layer was removed and the PBCECs were transferred to cell culture flasks. All cell lines were cultured in a humidified atmosphere containing 5% CO₂ at 37° C. and culture medium was renewed every other day unless the 80% confluence level was reached.

Quantification of cellular internalization in HeLa by flow cytometry. To quantify the cellular internalization of mRNA via flow cytometry, HeLa cells were seeded in a 96-well plate (VWR^{O}International, PA, USA) at a density of 10.000 cells/well (100 L/well) and left to settle overnight. After dilution in OptiMEM®, the cells were transfected with Cy5-labeled mRNA CADosomes, fLuc-mRNA control CADosomes or Cy5-labeled mRNA DOTAP-DOPE lipoplexes (100 ng mRNA/well) during 4 h at 37° C. in humidified atmosphere containing 5% CO₂. Following incubation, the cells were washed with PBS and harvested by trypsinization (trypsin/EDTA 0.25%). After neutralization in CCM, the cell suspensions were transferred to a U-bottom 96-well plate (Greiner Bio-One GmbH, Vilvoorde, Belgium), which was centrifugated during 5 min at 500 g. Next, the cells were resuspended in 80 L of flow buffer (PBS supplemented with 1% bovine serum albumin and 0.1% sodium azide) and kept on ice until flow cytometry analysis. The samples were analyzed using the CytoFLEX® flow cytometer (Beckman Coulter, Krefeld, Germany) and CytoExpert software. Data analysis was performed using the FlowJo™ analysis software (Version 10.5.3, Treestar, Costa Mesa, CA, USA). The Mean Fluorescence intensity (MFI) of the tested mRNA formulations was normalized to a non-treated cell population (NTC) to calculate relative-Mean Fluorescence intensity (rMFI) values.

Quantification of eGFP-mRNA expression in HeLa by flow cytometry. To quantify the expression of eGFP-mRNA via flow cytometry, HeLa cells were seeded in a 96-well plate (VWR© International, PA, USA) at a density of 10.000 cells/well (100 μL/well) and left to settle overnight. After dilution in OptiMEM®, the cells were transfected with eGFP-mRNA CADosomes, fLuc-mRNA control CADosomes or eGFP-mRNA DOTAP-DOPE lipoplexes (100 ng mRNA/well) during 4 h at 37° C. in humidified atmosphere containing 5% CO₂. Following incubation, the cells were washed with PBS and incubated with fresh CCM for 24 h. Next, flow cytometry sample preparation data acquisition and analysis was performed as previously described. Transfection yield was calculated based on the following equation: (rMFI eGFP*eGFP expression %)/(rMFI Cy5*Uptake %).

Visualization of cellular internalization and eGFP-expression in HeLa by confocal microscopy. HeLa cells were seeded in two separate black, cyclic olefin copolymer 96-well plates for high content imaging (PerkinElmer Health Sciences, Groningen, Nederland) at a density of 10.000 cells/well (100 μL/well) and left to settle overnight. To visualize Cy5-mRNA cytosolic internalization, Cy5-mRNA CADosomes (NT-DOPE) were added to the cells in OptiMEM® (100 ng Cy5-mRNA/well). Similar steps were performed to visualize eGFP expression using eGFP-mRNA CADosomes (100 ng eGFP/well). The cells were incubated for 4 h at 37° C. in humidified atmosphere containing 5% CO₂. Following incubation, the cells transfected with eGFP-mRNA CADosomes were washed with PBS and incubated with fresh CCM for 24 h until imaging, while the cells transfected with Cy5-mRNA CADosomes were fixated with 4% paraformaldehyde during 10-15 min at room temperature. Next, the nuclei were stained by adding one drop of VECTASHIELD® antifade mounting medium with DAPI per well (Vector Laboratories, CA, USA). The same procedure was applied on the cells containing eGFP-mRNA CADosomes after 24 h incubation. A laser scanning confocal microscopy (Nikon A1R HD confocal, Nikon, Japan), equipped with a 20× air objective lens (20×CFI Plan Apo VC, NA 0.75, WD 1000 μm, Nikon, Japan), with a laser box (LU-N4 LASER UNIT 405/488/561/640, Nikon Benelux, Brussels Belgium) and detector box (A1-DUG-2 GaAsP Multi Detector Unit, GaAsp PMT for 488 and 561 and Multi-Alkali PMT for 640 and 405 nm). The 405 nm, 488 nm and 640 nm laser were applied to excite the DAPI labeled nuclei, the eGFP protein and the Cy5-mRNA respectively. Fluorescence emission was detected through a 450/50 nm (MHE57010), 525/50 nm (MHE57030) and 700/75 nm (MHE57070) filter cube, respectively. A Galvano scanner was used for unidirectional scanning to acquire the channels sequential without line averaging and a scan speed of 0.042 FPS. The pinhole was set to 17.88 μm and the pixel size was 150 nm/pixel. NIS Elements software (Nikon, Japan) was applied for imaging.

Quantifying pharmacological activity of mRNA NT-DOPE CADosomes via a HEK293T cell Nanoluciferase Binary Technology (NanoBiT®) assay. To evaluate the pharmacological activity of the encapsulated nortriptyline (NT) in mRNA CADosomes, a previously described HEK293T (Human Embryonic Kidney) reporter cell line was used, stably expressing the 5-HT2AR (Serotonin 2A Receptor) and the cytosolic protein μ-arrestin 2 (βarr2) in the NanoLuciferase Binary Technology (NanoBiT®) system. The latter consists of two (inactive) split fragments of the NanoLuc enzyme (1 kDa SmBiT and 18 kDa LgBiT), each fused to one of the potentially interacting proteins (5-HT_{2A}R and βarr2). Binding of a receptor agonist, in this case LSD, results in βarr2 recruitment to the 5-HT2AR with the concomitant functional complementation of the enzyme, which can be monitored through the luminescence generated in the presence of the substrate. The HEK293T cells were seeded in poly-D-lysine (Sigma-Aldrich, Overijse, Belgium) coated 96-well plates at a density of 50.000 cells/well (100 L/well) and left to settle overnight. Next, the cells are rinsed with HBSS (Gibco©-Life Technologies, Grand Island, NY, USA) to remove remaining medium and serum, and 100 μL of the test solutions, containing mRNA CADosomes, free NT dissolved in HBSS (30 M and 100 M) or blank HBSS is placed on the cells, followed by another 4 h incubation in a humidified atmosphere at 37° C. and 5% CO₂. Subsequently, 25 μL of Nano-Glo® Live Cell Substrate (Promega, Madison, USA, diluted 1/20 in Nano-Glo© LCS Dilution Buffer, according to the manufacturer's protocol), is added to each well and the 96-well plate is transferred to a Tristar2 LB 942 multimode microplate reader (Berthold Technologies GmbH & Co, Germany). Upon stabilization of the luminescence signal, either concentrated LSD solution (Sigma-Aldrich, Overijse, Belgium) or the appropriate solvent controls are added, and the luminescence is continuously monitored for 2 h. The obtained real-time activation profiles are corrected for inter-well variability, followed by a calculation of the Area Under the Curve (AUC), from which the appropriate solvent control is deducted.

Quantification of cellular Cy5-labeled mRNA internalization and eGFP-expression in primary bovine corneal epithelial cells (PBCECs) by flow cytometry. To quantify the gene expression of eGFP-mRNA and Cy5-mRNA cellular uptake via flow cytometry, PBCECs were seeded in a 96-well plate (VWR© International, PA, USA) at a density of 25.000 cells/well (100 μL/well) and left to settle overnight. Next, the cells were washed with OptiMEM® before adding eGFP-mRNA or Cy5-labeled mRNA CADosomes and DOTAP-DOPE lipoplexes diluted in OptiMEM® (100 ng mRNA/well), during 4 h at 37° C. in humidified atmosphere containing 5% CO₂. Following incubation, the cells were washed with PBS and incubated with fresh CCM for 24 h. The cells were then prepared for flow cytometry analysis as described above for the quantification of eGFP expression and Cy5-mRNA cellular internalization in HeLa cells.

Cell Viability. HeLa cells (10.000 cells/well) and PBCECs (25.000 cells/well) were seeded in a 96-well plate and transfected with mRNA CADosome or DOTAP-DOPE lipoplexes. The cell viability was determined with the CellTiter-Glo© assay (Promega, Belgium). The culture plates and reconstituted assay buffer were placed at room temperature for 30 min, before initiating the assay. Subsequently, the CCM was replaced by 100 μL fresh CCM and an equal amount of assay buffer was added. To induce complete cell lysis, the plates were shaken during 2 min and the signal was allowed to stabilize the following 10 min. Next, 100 μL from each well was transferred to an opaque 96-well plate, which was measured with a GloMax© 96 Microplate Luminometer (Promega, Belgium).

Delivery of Cre-recombinase-mRNA in HeLa reporter cells by flow cytometry. To quantify eGFP expression after Cre-recombinase mediated elimination of the DsRed stop-codon, HeLa reporter cells were seeded in a 96-well plate (VWR® International, PA, USA) at a density of 10.000 cells/well (100 L/well) and left to settle overnight. Next, the cells were washed with OptiMEM® before adding eGFP-mRNA CADosomes and DOTAP-DOPE lipoplexes diluted in OptiMEM® (100 ng mRNA/well), during 4 h at 37° C. in humidified atmosphere containing 5% CO₂. Following incubation, the cells were washed with PBS and incubated with fresh CCM for 24 h. The cells were then prepared for flow cytometry analysis as described above for the quantification of eGFP expression in HeLa cells.

Preparation and physicochemical characterization of CAD-LNPs. CAD-LNPs were synthesized by injecting one volume of lipid mixture in different mol ratios of CAC, DLin-MC3-DMA (heptatriaconta-6,9,28,31-tetraen-19-yl 4-(dimethylamino)butanoate, abbreviated as MC3), DSPC (1,2-distearoyl-sn-glycero-3-phosphocholine), cholesterol and DMG-PEG2000 (1,2-dimyristoyl-rac-glycero-3-methoxypolyethylene glycol-2000) in ethanol and three volumes of siRNA (optimal molar N/P charge ratio of 4.7) in acetate buffer (pH 5, 10 mM) in the microfluidic NanoAssemblr® Benchtop mixing device (Precision Nanosystems, Vancouver BC, Canada) at a total flow rate of 12 mL/min (3 mL/min for ethanol and 9 mL/min for aqueous buffer, flow rate ratio of 3:1 (aqueous to ethanol)). The resultant mixture (5.8 mg/mL total lipid concentration) was dialyzed (Pur-A-Lyzer™ Maxi 12000 Dialysis Kit) overnight against phosphate buffered saline (PBS) to remove residual ethanol and to raise the pH to 7.4. MC3 and all other lipids were purchased from MedChemExpress® and Avanti Polar Lipids, Inc. (Alabaster, AL, USA) respectively. Samples were stored at 4° C. until use. Hydrodynamic diameter, zeta-potential and polydispersity index (PDI) of the MC3 LNPs (after dialysis) were determined in HEPES buffer (pH 7.4, 20 mM) via Dynamic Light Scattering (DLS) (Zetasizer Nano, Malvern Instruments Ltd., Worcestershire, UK). siRNA complexation and encapsulation efficiency were respectively determined by agarose gel electrophoresis and a Quant-iT™ RiboGreen® RNA assay.

Preparation and physicochemical characterization of CADosomes via microfluidic mixing technology. CADosomes were synthesized by injecting 16 μL of lipid mixture of CAC, DOPE (1,2-dioleoyl-sn-glycero-3-phosphoethanolamine), cholesterol and DMG-PEG2000 (1,2-dimyristoyl-rac-glycero-3-methoxypolyethylene glycol-2000) using different mole ratios in ethanol and 32 μL of eGFP-mRNA (10 μg, optimal N/P charge ratio of 6-10) in acetate buffer (pH 5, 25 mM), followed by 1:1 PBS-dilution, in the microfluidic NanoAssemblr© SPARK™ device (Precision Nanosystems, Vancouver, Canada). Standard settings and operation volumes were used to formulate the CADosomes as recommended by the SPARK™ user guide. The final mixture (2.1 mg/mL total lipid concentration) was dialyzed (Pur-A-Lyzer™ Mini 12000 Dialysis Kit) for at least 4 hours against phosphate buffered saline (PBS). CAC was purchased from Sigma-Aldrich (Overijse, Belgium) or Cayman Chemical (Michigan, USA). All other lipids were purchased from Avanti Polar Lipids, Inc. (Alabaster, Alabama, USA). Samples were stored at 4° C. until use. Hydrodynamic diameter, zeta-potential and polydispersity index (PDI) of the LNPs (after dialysis) were determined in HEPES buffer (pH 7.4, 20 mM) via Dynamic Light Scattering (DLS) (Zetasizer Nano, Malvern Instruments Ltd., Worcestershire, United Kingdom). To determine mRNA encapsulation efficiency, a Quant-iT™ RiboGreen® RNA assay (ThermoFisher Scientific) was used, according to manufactures instructions.

Statistical Analysis. All experiments were performed as technical triplicate and 3 independent biological repeats (n=3) unless otherwise stated. All data are presented as mean±standard error of the mean (SEM). Statistical analysis was performed using the 8th version of the GraphPad Prism software. One-way ANOVA with Tukey Correction was applied to compare multiple conditions, whereas the student t-test was used for direct comparison of 2 conditions. A p value ≤0.05 was considered a priori to be statistically significant (ns p>0.05, * p≤0.05, ** p≤0.1.* p≤0.0.** p≤0.0001).

### Results

### Preparing CADosomes Using the Tricyclic Antidepressant Nortriptyline as Structural Component.

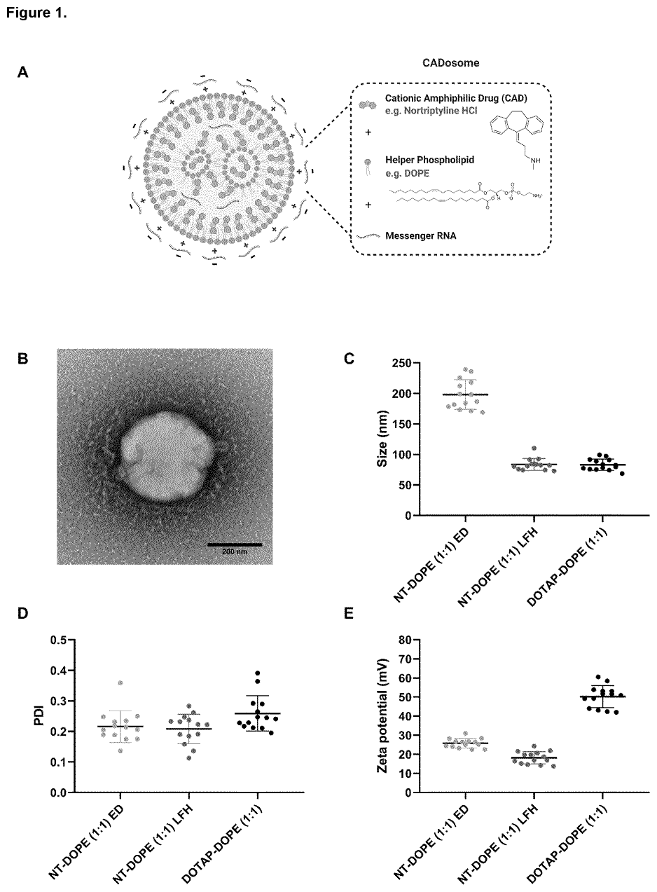

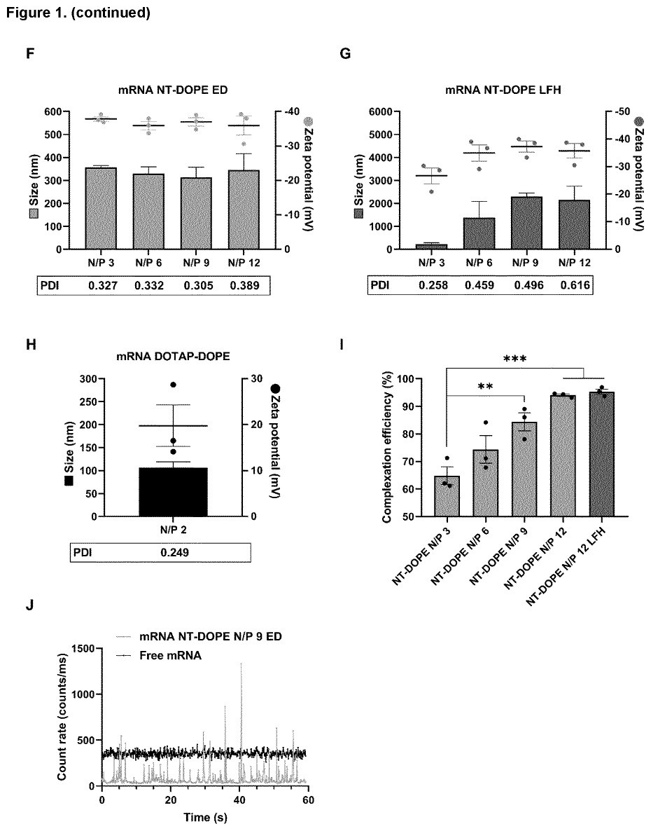

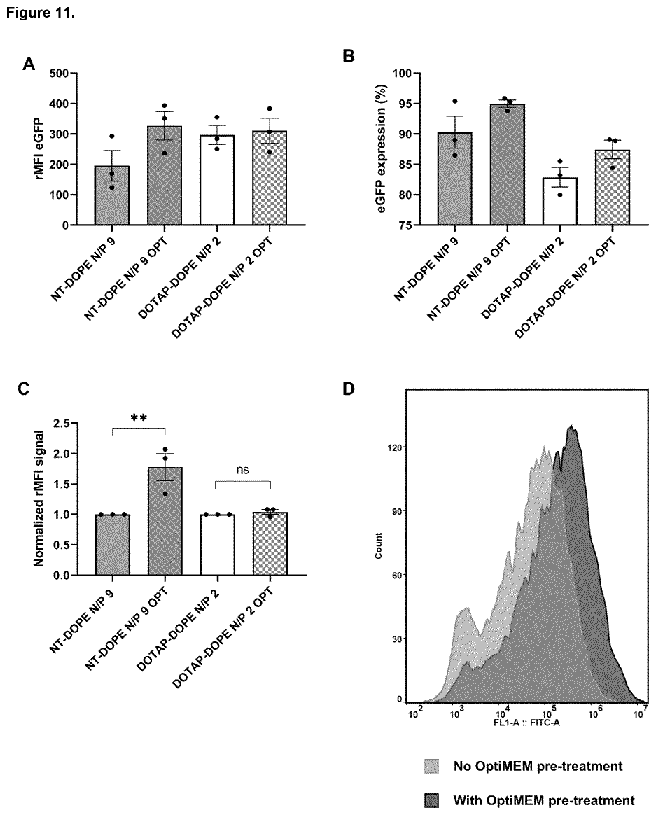

Previous work has illustrated that sequential administration of CADs to NP-transfected cells facilitates cytosolic delivery of small RNA therapeutics (e.g. siRNA) but not their larger counterparts (e.g. mRNA) (Joris et al. 2018; Van de Vyver et al. 2020). Here, we evaluated if CADs can be harnessed as structural and functional component of LNPs for mRNA complexation and delivery. With their charged headgroup, CADs can interact with anionic phosphatidylserine membranes via coulomb- or ion-induced dipole interactions, while zwitterionic phosphatidylcholine and phosphatidylethanolamine domains are mainly reached by Van der Waals forces and hydrophobic effects. TCA nortriptyline hydrochloride (NT) (c log P 4.51 and pKa 10.1) was initially used as model CAD molecule to explore NP formation in combination with the widely used helper lipid DOPE. Stable and cationic vesicles could be obtained by simply mixing NT with DOPE (50:50 molar ratio), indicating their self-assembly, both via ethanol dilution and lipid film hydration methods, as schematically illustrated in FIG. 1A. Lipid film hydration followed by sonication resulted in smaller NT-DOPE vesicles of ˜80 nm (PDI 0.2) with lower zeta potential (˜+20 mV) compared to ethanol dilution (˜200 nm (PDI 0.2) and +25 mV). However, relative to conventional cationic DOTAP-DOPE liposomes (83 nm (PDI 0.3) and +50 mV) both NT-DOPE vesicles were substantially less positively charged (FIGS. 1C, D and E). In a next step, these vesicles were used for complexation of enhanced green fluorescent protein-encoding messenger RNA (eGFP-mRNA) at mounting nitrogen-to-phosphate (N/P) ratios to form NT-DOPE mRNA complexes further referred to as CADosomes (FIGS. 1F, G and H). Transmission electron microscopy (TEM) typically showed spherical CADosomes with a dense lipid core (FIG. 1B and FIG. 8). CADosomes slightly increased in size when using ethanol dilution-derived NT-DOPE vesicles for mixing with mRNA, while the NT-DOPE vesicles obtained via lipid film hydration aggregated after mRNA complexation at all N/P ratios tested. Nevertheless, fluorescence correlation spectroscopy (FCS) analysis confirmed that up to 95% complexation efficiency could be reached at the highest N/P ratios tested (FIG. 11 and FIG. 1J). However, independent of the N/P ratio and vesicle preparation method, CADosomes shifted to a negative zeta potential, indicating that part of the mRNA strands is located on the outer surface of the complexes, while DOTAP-DOPE complexes remained positively charged at their most optimal N/P ratio. Altogether, these data indicate that NT in combination with DOPE self-assembles into cationic vesicles with high mRNA complexation efficiency.

Exploring Nortriptyline CADosomes for Cytosolic mRNA Delivery.

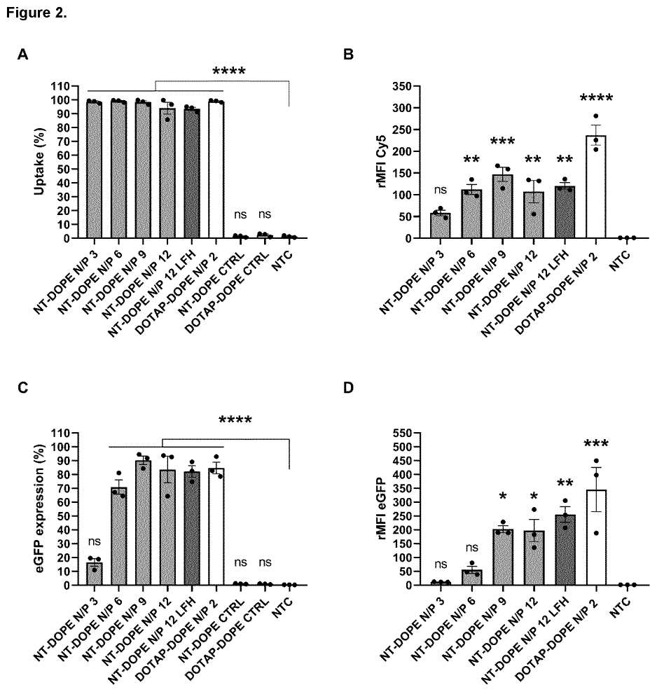

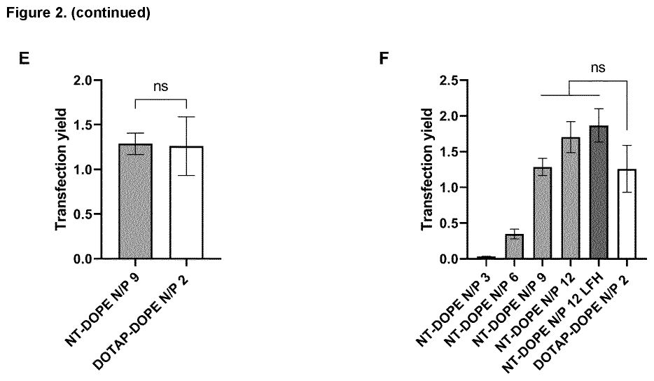

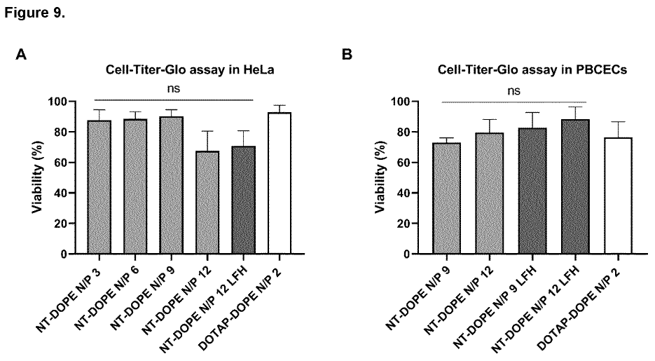

Having demonstrated that stable CADosomes could be formed with NT, we next aimed to evaluate in vitro mRNA delivery. Hereto, NT-DOPE CADosomes were first loaded with Cy5-labeled mRNA and their cellular uptake in HeLa cells was evaluated via confocal microscopy and flow cytometry (FIG. 2A-B). Independent of the NT-DOPE mRNA N/P ratio, >95% Cy5-positive cells was obtained (FIG. 2A). However, the relative mean fluorescence intensity (rMFI) compared to the non-treated control (NTC) was highest for CADosomes N/P 9, but remained significantly lower than DOTAP-DOPE (FIG. 2B). A possible explanation could be the difference in surface charge of both nanocarriers (FIG. 1F-H), where the negative zeta potential for the CADosomes is expected to reduce cell binding and internalization. Confocal microscopy confirmed the internalization of Cy5-mRNA-loaded CADosomes in transfected HeLa cells. Next, mRNA-induced eGFP expression using CADosomes was determined 24 h post-transfection as a function of the N/P ratio and compared with cationic DOTAP-DOPE lipoplexes (N/P 2) (FIGS. 2C-F). Up to 90% of the HeLa cells successfully expressed eGFP after transfection with CADosomes N/P 9 (FIG. 2C), which was visually confirmed via confocal microscopy. Furthermore, a higher N/P ratio correlated with a significant increase in eGFP expression (FIG. 2D). For the more stable CADosomes obtained via ethanol dilution, highest eGFP expression was observed for N/P 9, which was consequently selected as the optimal composition. Of note, taking into account the lower cellular uptake of mRNA obtained via transfection with CADosomes, an equivalent mRNA transfection yield (i.e. eGFP expression normalized to the intracellular mRNA dose) was reached relative to the DOTAP-DOPE lipoplexes (FIG. 2E-F). It has been demonstrated in the literature that the helper lipid DOPE, containing a small phosphoethanolamine headgroup and two unsaturated oleoyl chains, displays a cone-like geometry that promotes the non-bilayer, inverse hexagonal (Hii) phase during endolysosomal membrane fusion or bilayer disruption. Therefore, we hypothesize that upon cellular internalization DOPE contributes to the cytosolic delivery of mRNA via membrane fusion, while the CAD molecules are mainly responsible for mRNA complexation. Lastly, a Cell-Titer-Glo® viability assay demonstrated acceptable cell viability for CADosomes N/P 3 to 9, while N/P 12 resulted in ˜35% cell death in HeLa cells but not in PBCECs (FIG. 9).

Screening of CADosomes with Diverging CADs for mRNA Delivery in HeLa Cells

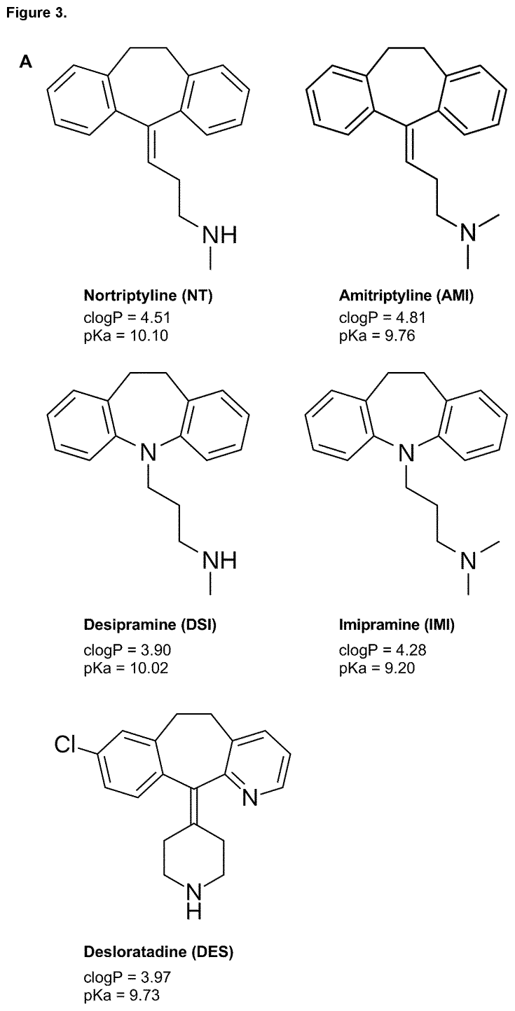

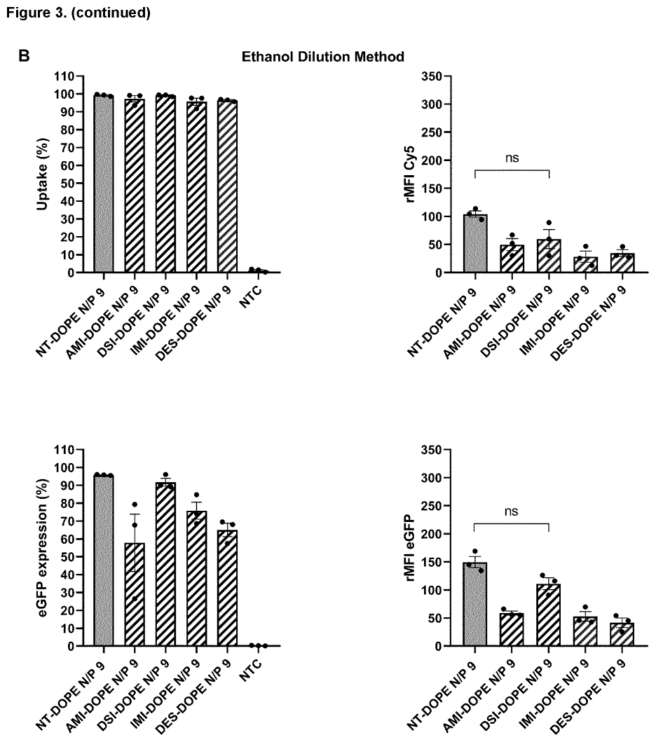

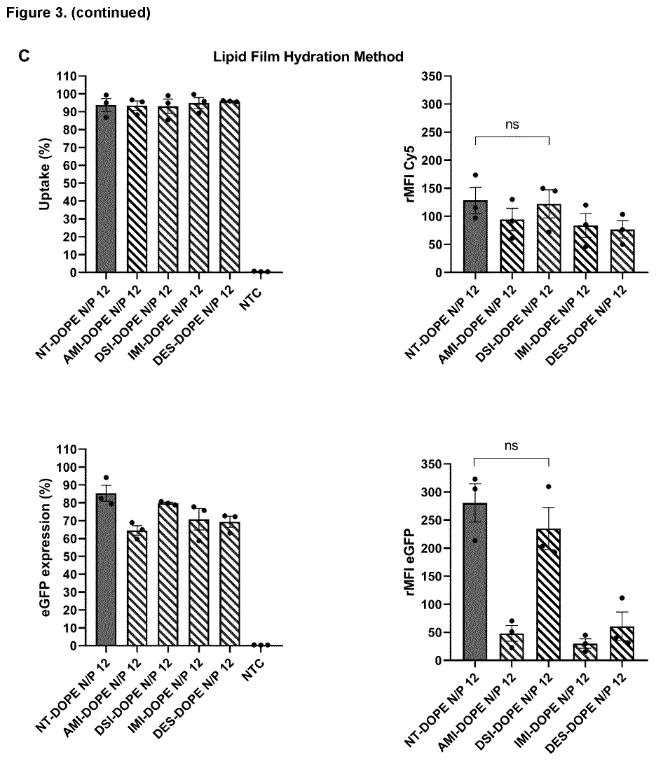

To evaluate if our CADosome strategy for mRNA delivery could be extended to other CAD molecules, a small screen of selected CAD compounds (Table 2) were likewise mixed with DOPE (50:50 ratio). Ketotifen (c log P 3.35 and pKa 7.15), verapamil (c log P 5.04 and pKa 9.68) and epinastine (c log P 3.13 and pKa 9.31) did not self-assemble with DOPE to form stable nanosized cationic vesicles, neither via ethanol dilution nor lipid film hydration (data not shown). On the other hand, successful vesicle formation was achieved with amitriptyline hydrochloride (AMI) (c log P 4.81 and pKa 9.76), desipramine hydrochloride (DSI) (c log P 3.90 and pKa 10.02), imipramine hydrochloride (IMI) (c log P 4.28 and pKa 9.20), and desloratadine (DES) (c log P 3.97 and pKa 9.73), as illustrated in FIG. 3. The latter CADs are structurally related to NT and are composed of an aromatic tricyclic domain with a three carbon tail substituted with secondary- or tertiary methylated amine groups, or a heterocyclic amine piperidine group in case of desloratadine. This cationic lipid-like structure, with clearly segregated hydrophobic and charged amine moieties, could explain this self-assembly behavior with DOPE, in contrast to loperamide, verapamil and epinastine. The lower pKa value of ketotifen compared to the other compounds suggests the importance of a positive charge to electrostatically interact with the polar groups of phospholipids and enable membrane insertion. Next, cellular uptake of Cy5-mRNA in HeLa cells was quantified for the respective CADosomes, showing >90% Cy5-positive cells independent of the production method and CAD type (FIG. 3B-C). While all tested CAD types showed an acceptable mRNA internalization efficiency, only desipramine (DSI)-DOPE showed comparable mRNA internalization efficiency to NT-DOPE, in contrast to their CADosome counterparts with amitriptyline and imipramine, only differing in the substitution degree of the amine group. This trend was also reflected in the eGFP expression levels, with DSI-DOPE showing similar mRNA delivery efficiency to NT-DOPE, reaching up to 90% eGFP+ cells with high expression levels (FIG. 3B-C). Also CADosomes based on DES, with a secondary amine concealed in a more bulky piperazine ring structure, are less efficient.

Evaluation of the Pharmacological Activity of CADosomes Using Nanoluciferase Binary Technology (NanoBiT®)

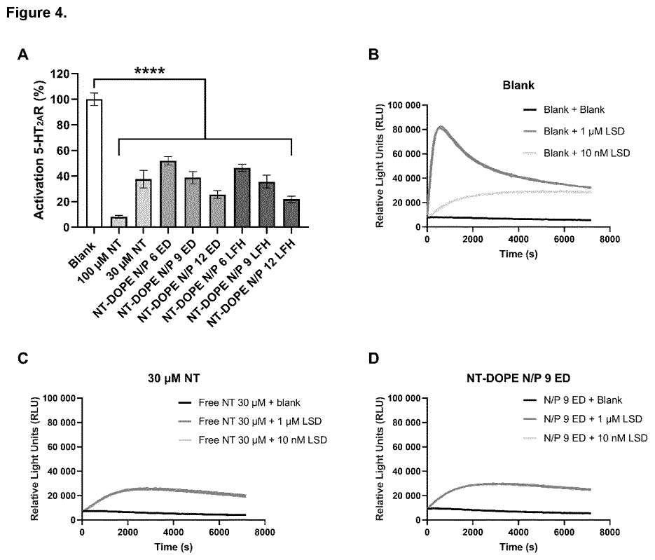

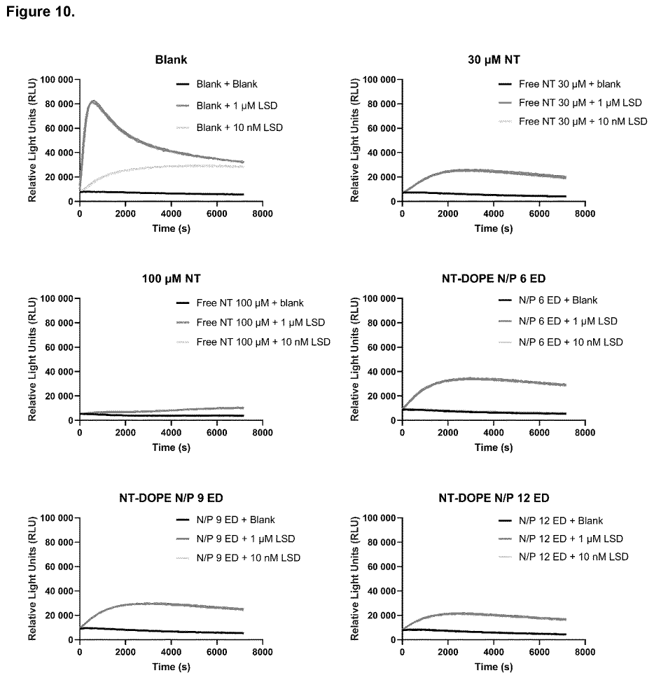

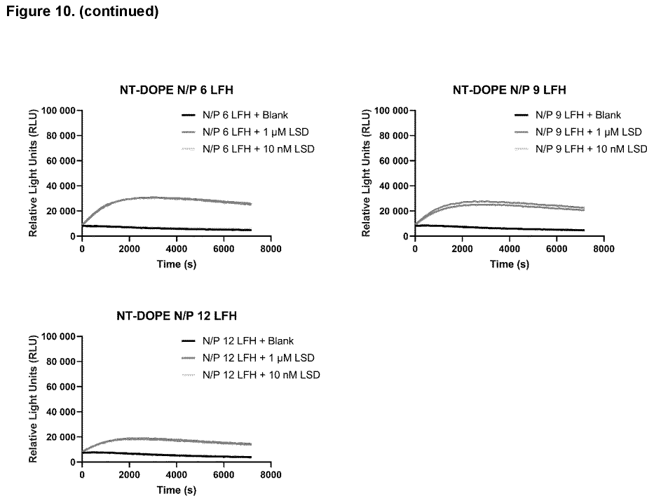

CADosomes that are able to deliver both a functional CAD and therapeutic mRNA in one single NP formulation would provide ample opportunities for combination therapy. To assess if CADs remain pharmacologically active after incorporation in CADosomes, a previously described luminescence bioassay was used, based on HEK293T (Human Embryonic Kidney) cells stably expressing the 5-HT2AR (serotonin 2A receptor) and the cytosolic protein μ-arrestin 2 (βarr2) in the Nanoluciferase Binary Technology (NanoBiT©) system. Binding of a 5-HT2AR receptor agonist, in this case lysergic acid diethylamide (LSD), results in βarr2 recruitment to the 5-HT2AR and the concomitant functional complementation of the luciferase enzyme, which can be monitored through the luminescence generated in the presence of the substrate. Vice versa, binding of an antagonist to the 5-HT2AR, e.g. nortriptyline (NT), will hamper agonist-induced βarr2 recruitment and the formation of a luminescence signal. As expected, increasing concentrations of free NT (30 μM and 100 μM), reduced or even completely prevented 5-HT2AR activation by 1 μM, respectively 10 nM, of the agonist LSD (FIG. 4A). Furthermore, independent of the production method, transfecting the HEK293T cells with NT-DOPE CADosomes impaired LSD-induced 5-HT2AR activation with increasing N/P ratio, corresponding with a higher CAD content. Remarkably, no significant difference could be observed in 5-HT2AR antagonism between 30 μM of free NT and NT-DOPE CADosomes N/P 9, which contain the equal antagonist concentration. The obtained real-time receptor activation profiles clearly indicate similar trends (FIGS. 4B, C and D and FIG. 10). Since it was clearly demonstrated in FIG. 1 that NT is integrated in the lipid vesicles, leading to a positive surface charge and enabling mRNA complexation, these data suggest that the pharmacological activity of the TCA nortriptyline remains unaffected after CADosome incorporation.

CADosome-Mediated Cre-Recombinase mRNA Delivery

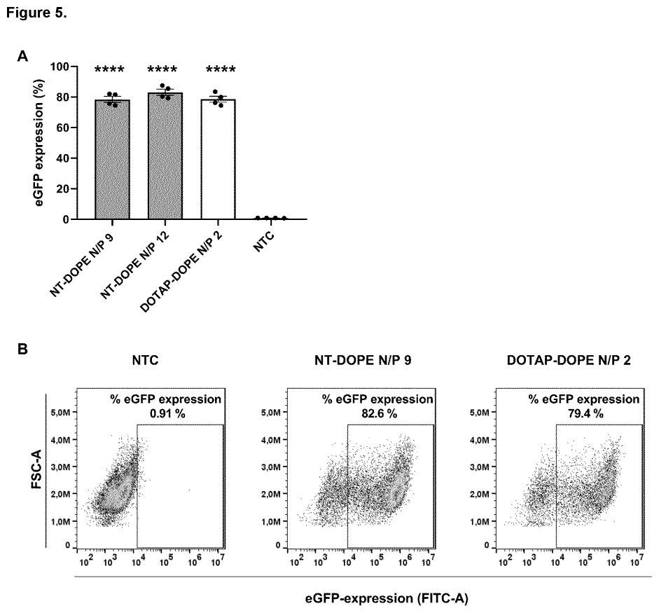

Next, a HeLa reporter cell line stably expressing the Cre reporter plasmid pLV-CMV-LoxP-DsRed-LoxP-eGFP was used to further demonstrate the delivery efficiency of CADosomes as nanocarriers of Cre-recombinase-encoding mRNA. Cre-recombinase is a tyrosine recombinase enzyme that is able to excise the DsRed stop-codon between loxP sites, causing a shift from red (DsRed+) to green (eGFP+) fluorescence in the reporter cells. Both NT-DOPE CADosomes N/P 9 and N/P 12 reached up to 80% eGFP+ cells 24 h post-transfection, as measured via flow cytometry. Interestingly, equal recombination efficiency was obtained as with cationic DOTAP-DOPE lipoplexes (FIG. 5A). A clear cell population shift could be observed on representative dot-plots of CADosomes N/P 9 (82.6% eGFP+ cells) and DOTAP-DOPE N/P 2 (79.4% eGFP+ cells) compared to non-treated cells (NTC) (FIG. 5B). These data indicate the versatility of CADosomes to deliver functional mRNA cargo's in a highly effective manner in line with conventional cationic lipoplexes.

CADosome-Mediated mRNA Delivery in Hard-to-Transfect Primary Bovine Corneal Epithelial Cells

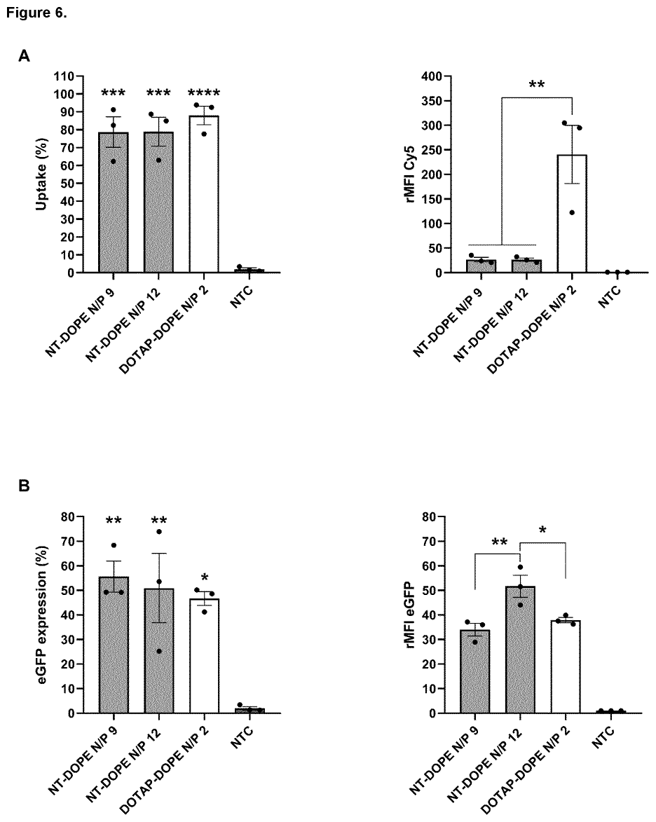

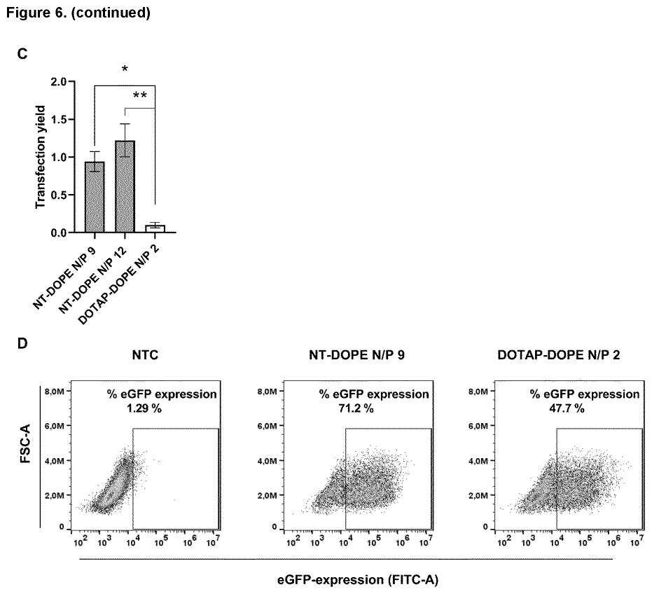

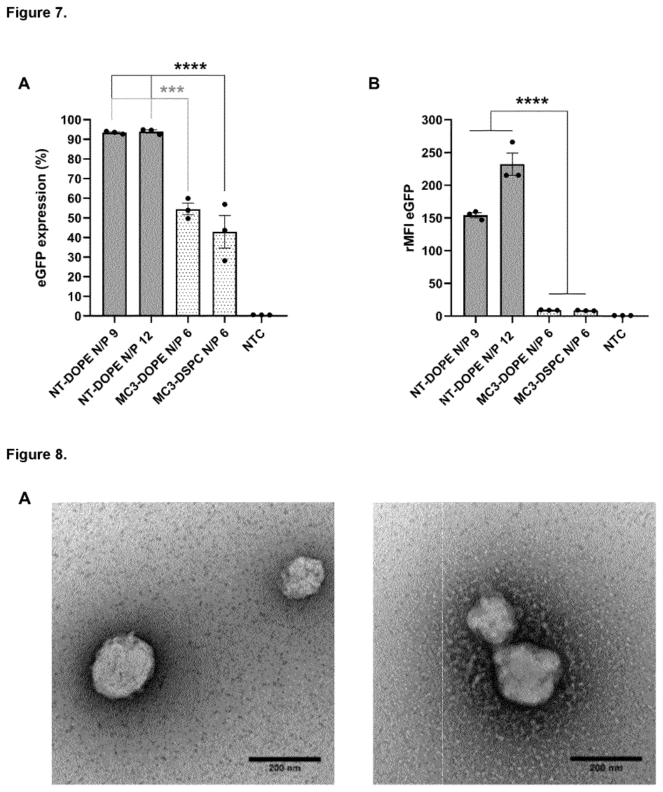

Having established that NT-DOPE CADosomes are able to deliver mRNA in HeLa cells, we next sought to evaluate their delivery performance in a more difficult-to-transfect primary cell type. As FIG. 1 indicated that at least a fraction of the mRNA will likely be exposed at the surface of the particles, we first further optimized our transfection protocol to reduce the risk of mRNA degradation during transfection. Raes et al., 2020, recently demonstrated that an additional washing step with OptiMEM before adding mRNA to the cells allowed to remove degradative enzymes from the culture medium and improved transfection efficiency with a physical delivery method. Applying this protocol to CADosomes, the eGFP expression likewise increased almost two-fold in HeLa cells, outperforming the transfection efficiency of DOTAP-DOPE lipoplexes (FIG. 11A-D). Next, freshly excised bovine eyes were collected at a local slaughterhouse and the corneal epithelial layer was separated from corneal stroma. The obtained primary bovine corneal epithelial cells (PBCECs) were cultured and subsequently treated with NT-DOPE CADosomes, loaded with a Cy5-labeled or eGFP-encoding mRNA. Despite 80% of the PBCECs demonstrating successful mRNA internalization following NT-DOPE transfection, the extent of cellular uptake was ˜10-fold lower compared to the cationic DOTAP-DOPE lipoplexes (FIG. 6A). Again, the difference in surface charge between both complexes could be responsible for this observation. Surprisingly, even with strongly reduced cellular internalization, the resulting eGFP expression 24 h post-transfection was highest for NT-DOPE CADosomes, reaching almost 55% eGFP positive PBCECs with >30% higher eGFP expression than the DOTAP:DOPE positive control (FIG. 6B-D). Of note, taking into account the lower cellular uptake of mRNA obtained via transfection with NT-DOPE CADosomes N/P 12, a ten-fold higher mRNA transfection yield was reached relative to the DOTAP-DOPE lipoplexes (FIG. 6C). Furthermore, treatment with CADosomes at higher N/P ratio did not affect PBCEC cell viability in contrast to previously described experiments in HeLa cells (FIG. 9). As illustrated via representative flow cytometry dot-plots (FIG. 6D), a significant cell population shift was observed for NT-DOPE N/P 9 compared to NTC, indicating a high transfection efficiency. The abovementioned data proposes CADosomes as a suitable carrier for mRNA delivery in human ex vivo explant models or for in vivo application to the cornea. Notably, in this hard-to-transfect cell type CADosomes NT-DOPE N/P 9 and N/P 12 markedly outperformed state-of-the-art ionizable lipid MC3-LNPs, reaching >90% eGFP+ cells while only about half of the cells were transfected with MC3-LNPs. In addition, CADosomes demonstrated >20-fold higher eGFP expression levels (FIG. 7). Such high expression levels for CADosomes also exceed what was reported earlier, which could be explained by donor variability in the PBCECs, which were now obtained from another bovine eye.

Physicochemical Characterization of siRNA Loaded CAD-LNPs

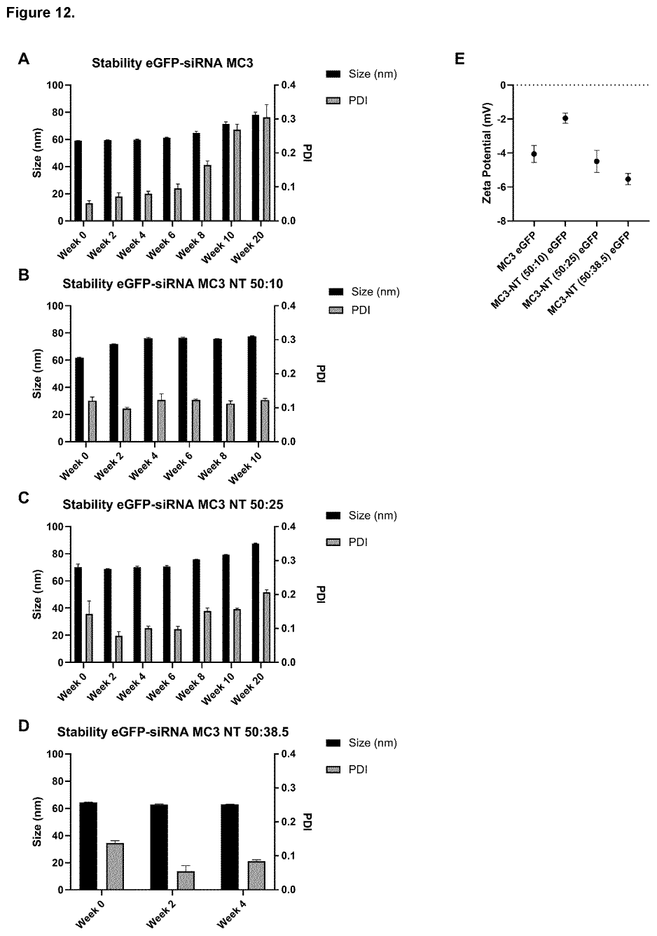

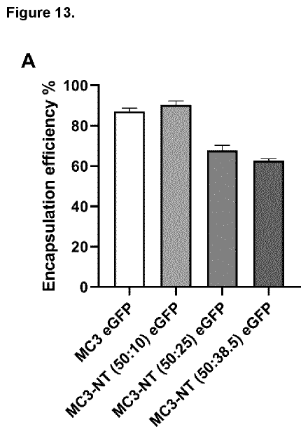

In this work, the state-of-the-art Onpattro formulation, containing DLin-MC3-DMA (MC3) was used as reference to produce CAD-LNPs via one-step microfluidic mixing technology. A top-down approach was implemented replacing the cholesterol fraction with the CAD molecule nortriptyline. Stability of the obtained formulations was measured over time via dynamic light scattering, as depicted in FIG. 12 A-E. The size of MC3-NT (10 mol % NT) LNP was around 60 nm with acceptable PDI<0.2 and almost neutral zeta potential. The particles remained stable up to 10 weeks after production. Similar trend could be observed with increasing mol % of nortriptyline (FIG. 12 C-E). The encapsulation efficiency measured via a Quant-iT™ RiboGreen® RNA assay (ThermoFisher Scientific) slightly decreased with lower cholesterol content. Nevertheless, >60% encapsulated siRNA was considered acceptable for CAD-LNPs (FIG. 13).

Quantification of MC3-NT LNPs eGFP-siRNA Knockdown Efficiency in H1299-eGFP Cell Line.

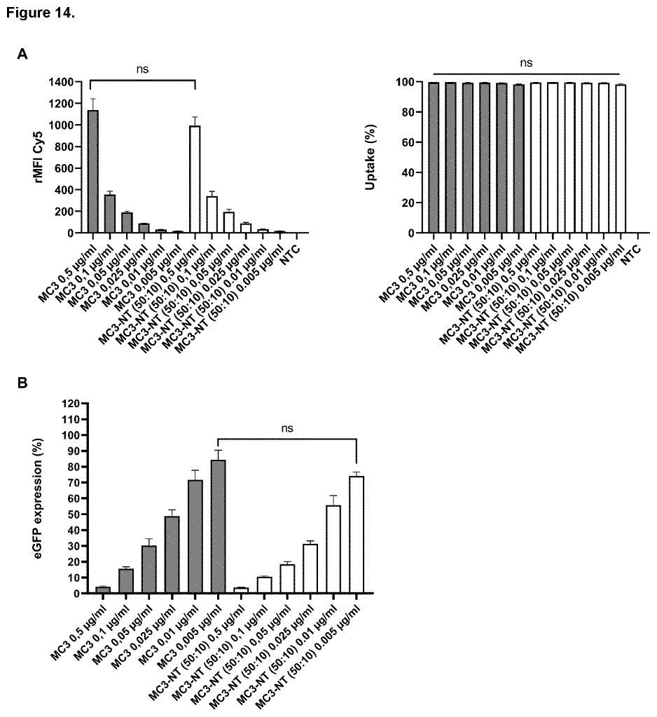

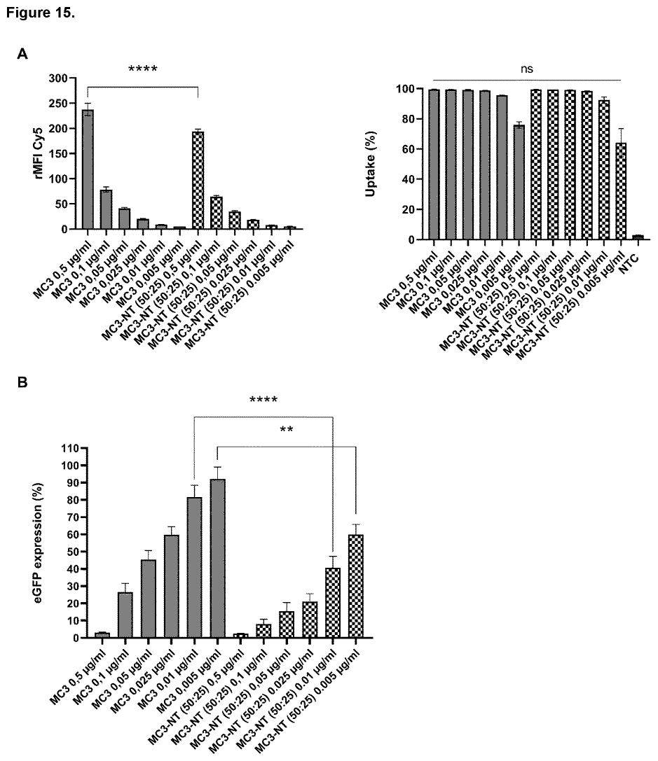

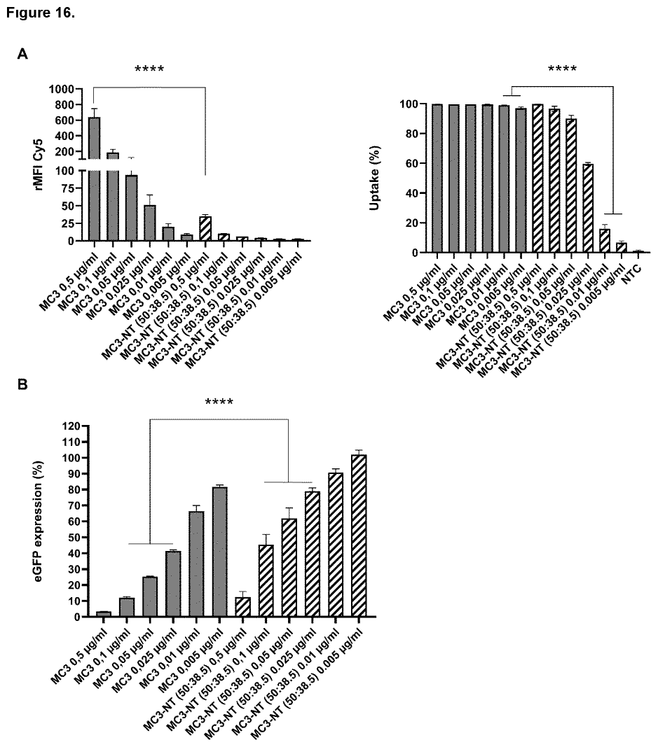

In FIG. 14, cellular uptake of MC3 LNPs and MC3-NT (50:10 mol %) LNPs, loaded with Cy5-labeled siRNA, in H1299-eGFP cells was analysed via flow cytometry. No significant difference in relative mean fluorescence intensity (rMFI Cy5; normalized to non-treated control (NTC)), could be observed. eGFP knockdown efficiency remained unchanged. Nevertheless, MC3-NT (50:25 mol %) LNPs outperformed the state-of-the-art MC3 LNPs, reaching better eGFP knockdown at lower siRNA concentrations (FIG. 15 A-B). Full replacement of the cholesterol fraction, as represented by the CAD-LNP formulation MC3-NT (50:38.5 mol %), resulted in significant lower Cy5-siRNA cellular internalization (FIG. 16 A). Furthermore, knockdown efficiency of MC3-NT (50:38.5 mol %) was also reduced (FIG. 16 B). Selective replacement of cholesterol by nortriptyline also enables improved intracellular delivery of mRNA, as was quantified in HeLa cells, relative to both the parent MC3 LNPs as well as MC3-NT LNPs (25 mol % NT) with proportional reduction in mol % of all other lipid components (data not shown).

Evaluating cholesterol-reduced siRNA CAD-LNPs with different CADs

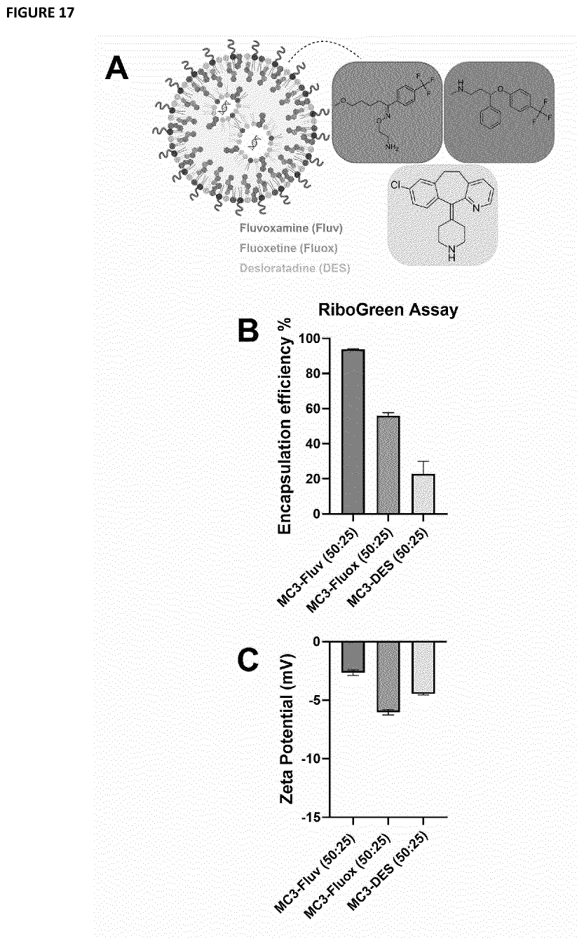

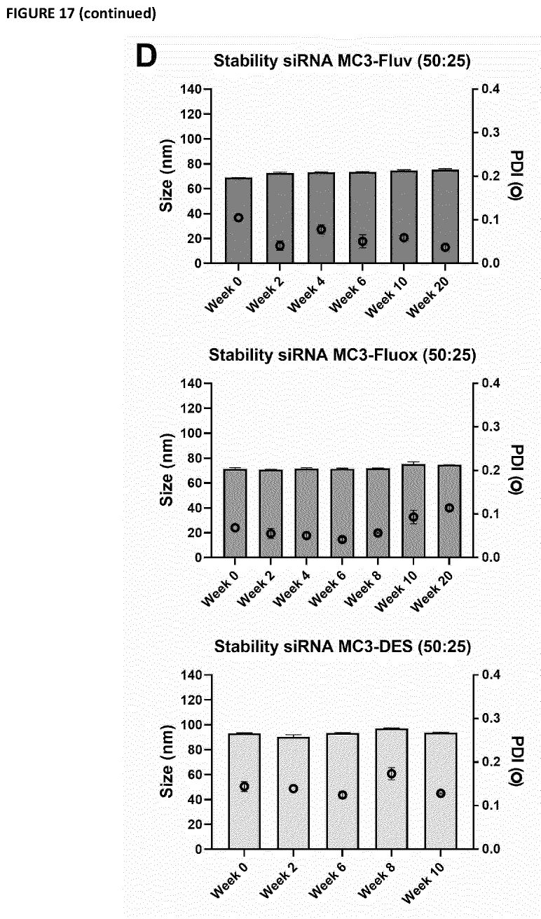

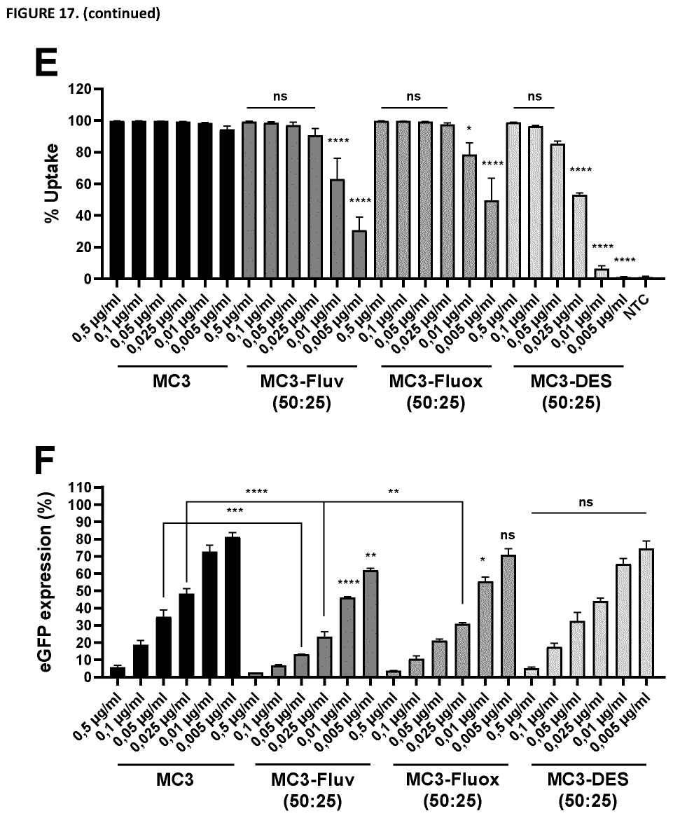

We previously demonstrated that replacing part of the cholesterol fraction of MC3-LNPs with the compound nortriptyline hydrochloride (NT) resulted in both enhanced siRNA-induced gene silencing as well as mRNA-encoded protein expression. Here, we evaluated a selection of other CAD molecules with diverging physicochemical properties for siRNA CAD-LNP formation and cytosolic delivery efficiency. Besides NT, a typical CAD molecule with an aromatic tricyclic domain substituted with a secondary amine group, also an antihistamine with a heterocyclic amine piperidine was investigated (DES) (c log P 3.97; pKa 9.73), as well as antidepressant CADs with monocyclic domains (Fluox and Fluv) of which the latter contains a primary amine group (c log P 4.17 and 2.89; pKa 9.8 and 8.86) (FIG. 17 A). Despite Fluv being the least hydrophobic compound tested, the siRNA encapsulation efficiency measured after microfluidic production of cholesterol-reduced MC3-Fluv (50:25) was >90%, while MC3-Fluox (50:25) reached almost 60%, comparable to MC3-NT (50:25) LNPs. Although DES resembles the chemical structure of NT most closely, MC3-DES (50:25) LNPs did not encapsulate siRNA well, showing <25% encapsulation efficiency (FIG. 17 B). The hydrodynamic diameter of MC3-Fluv (50:25) and MC3-Fluox (50:25) remained stable ˜70 nm with low PDI values (<0.2) up to 20 weeks after formulation, stored in PBS at 4° C. Zeta potential values were again close to neutral, comparable to MC3 parent LNPs (FIG. 17 C-D). In contrast to MC3-NT (50:25), significantly lower cellular uptake percentages were observed in H1299-eGFP cells for both MC3-Fluv (50:25) and MC3-Fluox (50:25) LNPs, but the reduced percentage was most outspoken for MC3-DES (50:25) LNPs (FIG. 4e). Nevertheless, eGFP silencing was still improved for both MC3-Fluv (50:25) and MC3-Fluox (50:25) LNPs relative to MC3-LNPs. Altogether, these findings demonstrate that CADs with distinct chemical structure are useful for the design of CAD-LNPs, illustrating the broader applicability of such formulations and enabling drug combination therapy for a variety of diseases. Of particular interest in the context of RNA inhalation therapy, Fluv has shown to interact with both the sigma-1 receptor and the serotonin transporter, which respectively influence viral replication of SARS-CoV-2 and facilitate anti-inflammatory effects. Additionally, NT has been repurposed as an anti-inflammatory agent by inhibiting the release of pro-inflammatory cytokines and restoring sensitivity to inhalation corticosteroids. Furthermore, desloratadine was repurposed for non-small cell lung cancer, enhancing the anti-neoplastic response of distinct chemotherapeutics.

In Vivo Biodistribution and Toxicity Analysis of siRNA-Loaded CAD-LNPs Following Intranasal Administration

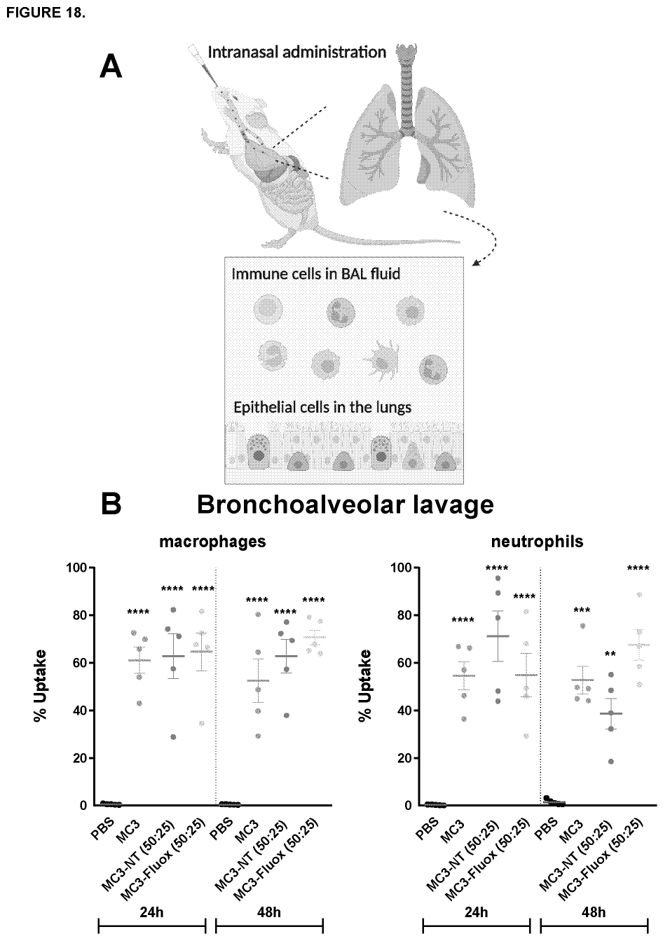

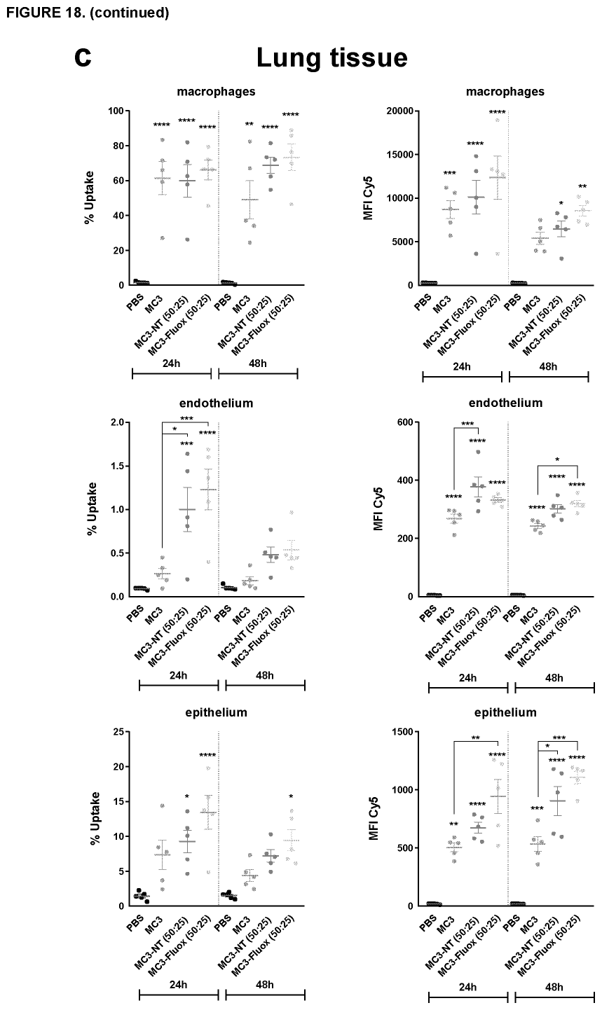

Next, we aimed to investigate which pulmonary cell types could be reached with cholesterol-reduced MC3-NT and MC3-Fluox (50:25) LNPs following intranasal administration in mice (FIG. 18 A). Macrophages were the most abundant cell type present in bronchoalveolar lavage (BAL) fluid and both macrophages and neutrophils scavenged most of the Cy5-siRNA LNPs within 24 h post-administration in C57BL/6 mice (FIG. 18 B). Alveolar macrophages represent the largest cellular fraction in the alveoli and are mainly responsible for phagocytic clearance of foreign material. Interestingly, in lung tissue, MC3-NT (50:25) and especially MC3-Fluox (50:25) LNPs, containing 25 mol % of the antidepressant fluoxetine instead of nortryptiline, reached the pulmonary epithelium and endothelium significantly better compared to non-modified MC3 LNPs (FIG. 18 C). Although the percentage of Cy5+epithelial cells decreased at later time points, the intracellular Cy5-siRNA dose remained significantly higher (>2-fold) compared to MC3 LNPs. Judging from pro-inflammatory cytokine response and immune cell infiltration following intranasal administration, it could be concluded that both state-of-the-art MC3-LNPs and modified CAD-LNPs did not induce marked pro-inflammatory effects in C57BL/6 mice.

Physicochemical Characterization of mRNA-Loaded CADosomes Produced Via One-Step Microfluidic Mixing Technology

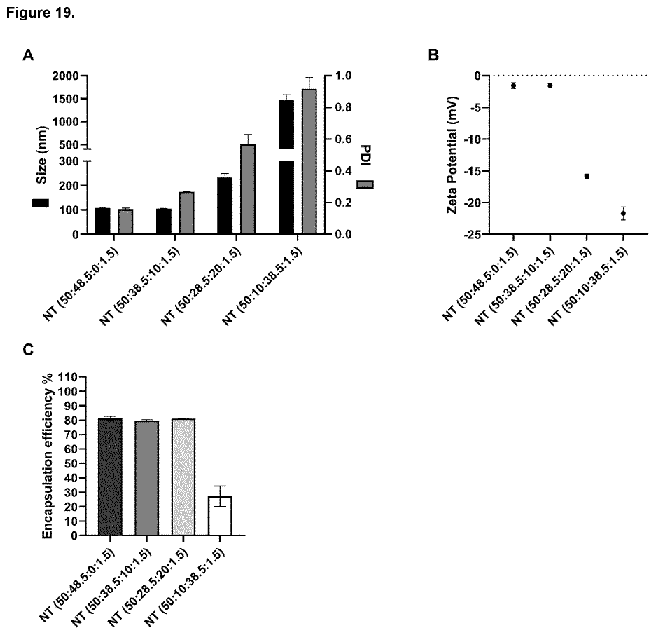

Having established that stable CADosome formulations could be formed via two-step ethanol dilution or classically used thin film hydration methods, we evaluated CADosome formulation using microfluidic mixing technology. Formulations containing Nortriptyline (NT), DOPE, cholesterol and DMG-PEG in different molar ratios (N/P 10) were prepared via the NanoAssemblr SPARK™ (Precision NanoSystems). The hydrodynamic diameter (Z-average) and polydispersity (PDI) of the resulting microfluidic CADosomes increased with higher cholesterol content. In contrast, zeta potential values slightly decreased, possibly indicating a higher free—or less encapsulated mRNA fraction (FIG. 19 A-B). This could be confirmed via a Quant-iT™ RiboGreen® RNA assay (ThermoFisher Scientific) where CADosome formulation containing NT and 38.5 mol % of cholesterol (50:10:38.5:1.5 mol %) encapsulated <30% mRNA (FIG. 19 C). Nevertheless, even without the presence of an ionizable lipid, stable CADosome formulations could be obtained under the given experimental conditions, encapsulating >80% mRNA.

Evaluation of mRNA Loaded Nortriptyline (NT)-CADosomes for Cytosolic Delivery of eGFP-Encoding mRNA (100 ng/Well) in a HeLa Cell Line.

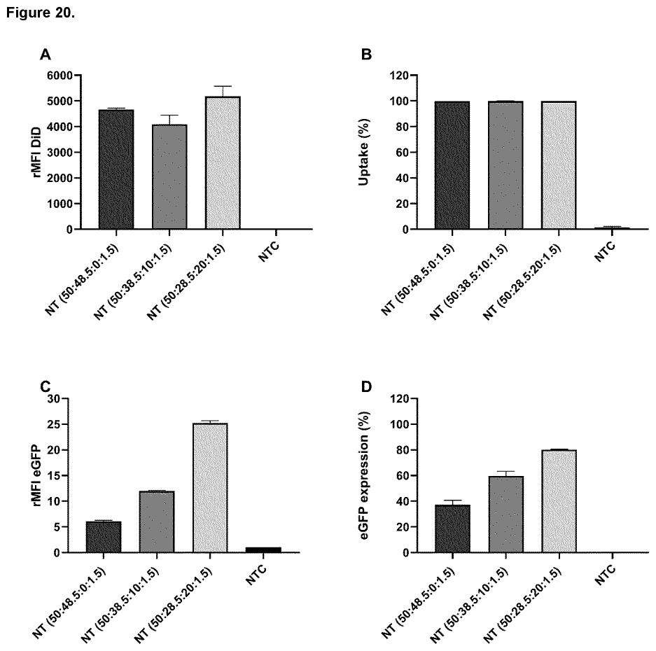

The physicochemically most stable CADosome formulations were further investigated for in vitro delivery of eGFP-encoding mRNA. CADosomes containing NT-DOPE-Chol-DMG-PEG with different molar ratios, loaded with a DiD-dye, were administered after dilution in complete cell culture medium on HeLa cells. Cellular uptake of DiD-labeled CADosomes were analyzed via flow cytometry 4 h post administration, as depicted in FIG. 20 A-B. Efficient internalization occurred independent of the CADosome's molar ratio. However, eGFP-expression slightly increased with higher cholesterol content, reaching almost 80% eGFP+HeLa cell population, with highest MFI values for the NT (50:28.5:20:1.5 mol %) CADosome (FIG. 20 C-D). This data indicate that the CADosome approach could be translated from a two-step production to more standardized one-step microfluidic production methods, while remaining biological active.

### Physicochemical Characterization of Different CADosomes Via Microfluidic NanoAssembir SPARK™

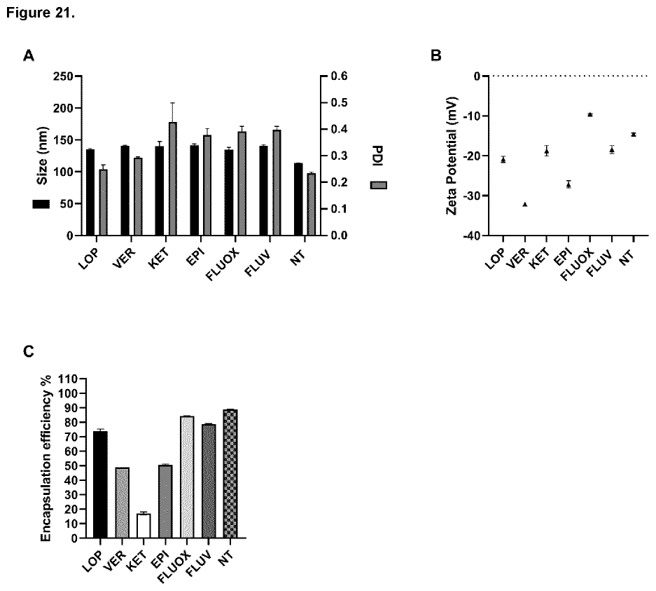

Having established that CAD molecules such as nortriptyline (NT), containing a tricyclic structure, were able to form stable CADosomes via microfluidic production, our goal was to further investigate if other CAD molecules with non-tricyclic structures could likewise form CADosomes. We selected loperamide HCl (LOP), verapamil HCl (VER), ketotifen fumarate (KET), epinastine HCl (EPI), fluoxetine HCl (FLUOX), fluvoxamine maleate (FLUV), as CAD molecules. Besides the CAD molecule, formulations contained DOPE, cholesterol and DMG-PEG (50:38.5:10:1.5 mole ratio; N/P 6) and were loaded with eGFP-encoding mRNA. Evaluation of hydrodynamic diameter (Z-Average diameter), PDI and zeta potential, measured via Dynamic Light Scattering (DLS), indicated that all tested CADs were able to form nanoparticles with acceptable size and stability (FIG. 21 A-C). The PDI value, especially of the Ketotifen (KET) CADosome was higher compared to NT reaching >0.4. Encapsulation efficiency of mRNA CADosomes measured via Quant-iT™ RiboGreen® Assay (InvitroGen), proved that mRNA encapsulation efficiency of LOP, FLUOX and FLUV CADosomes was >70%.

To conclude, this data indicate that besides tricyclic cationic amphiphilic drugs, as previously investigated for two-step CADosome formation, one step microfluidic CADosome production is also feasible with other CAD chemistries (e.g. with the basic amine more central in the molecular structure, molecules without the typical tricyclic structure, molecules with primary amines, . . . ).

mRNA CADosomes with Low Fractions of Ionizable Lipids

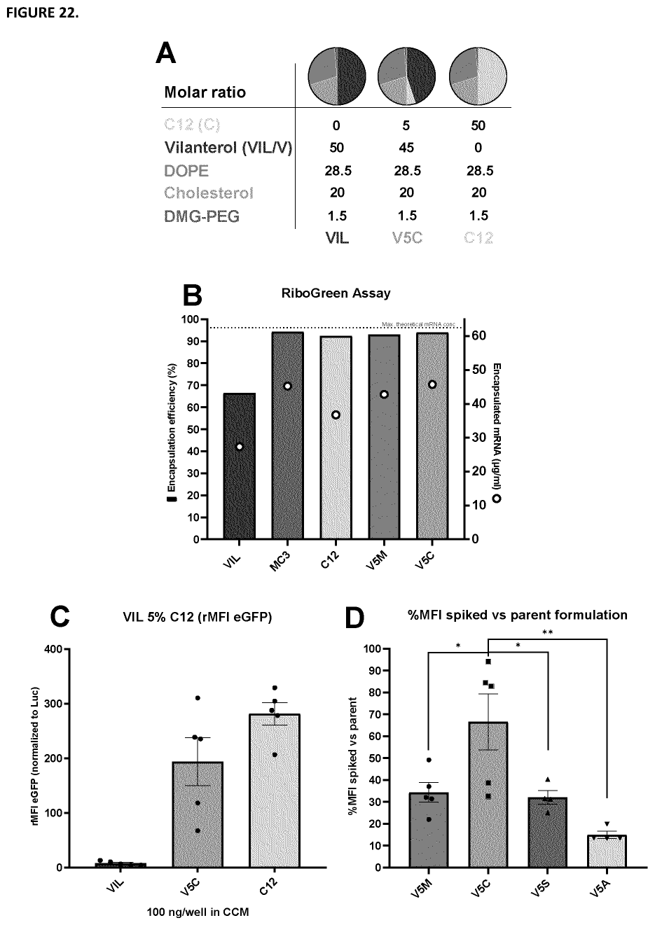

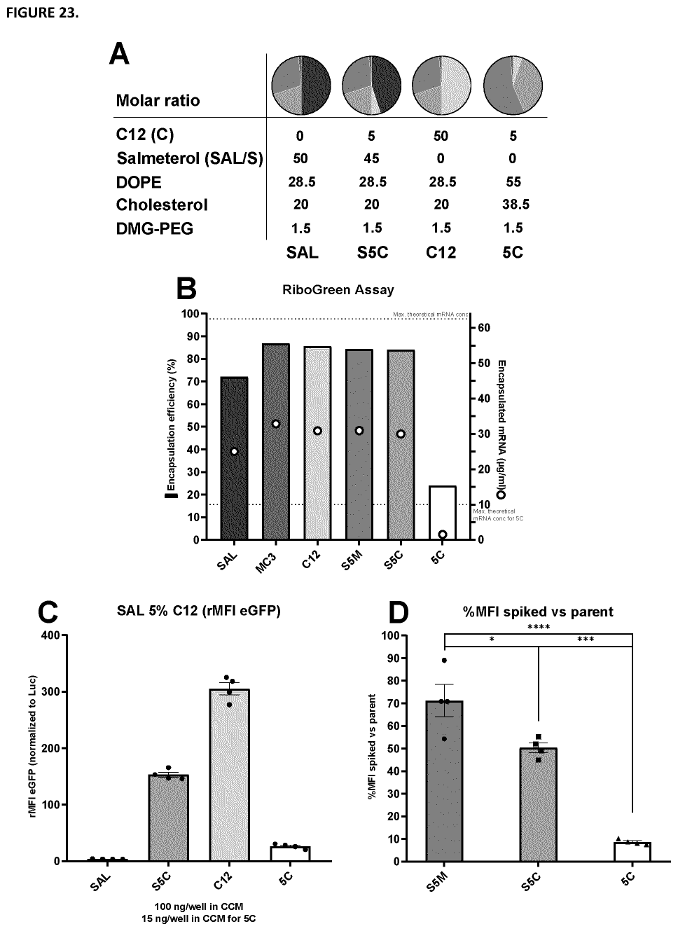

Here, the influence of spiking eGFP-encoding mRNA CADosomes with low molar fractions of ionizable lipids on both physicochemical properties and in vitro delivery efficiency was investigated. Hereto, the (manual) vortex mixing method was applied. FIGS. 22 A and 23 A show the composition of the formulations tested. Addition of 5 mol % fractions of ionizable lipids to CADosomes clearly demonstrated improved mRNA encapsulation efficiencies compared to the LNPs containing only CADs as cationic ionizable component (in casu the long acting beta-agonists vilanterol and salmeterol, FIGS. 22 B and 23 B). When applied to the immortalized human bronchial epithelial cell line BEAS-2B, a marked increase in eGFP expression was seen for the spiked formulations relative to the CAD-only LNPs (FIG. 22 C and FIG. 23 C), while maintaining up to 70% of the eGFP expression levels of the parent LNPs containing only the ionizable lipid. Of note, LNPs composed of only 5% of ionizable lipid did neither demonstrate acceptable mRNA encapsulation efficiencies (FIG. 23 B), nor biological activity (FIG. 23 C-D). These data indicate that spiking CADosomes with low fractions of ionizable lipid can provide hybrid formulations with high mRNA delivery potential. Such hybrid formulations with repurposed CADs as dominant component enable drug combination with excellent mRNA transfection capability while minimizing the need of cationic ionizable lipids, thus reducing cost and increasing biocompatibility.

### REFERENCES

Aits, S.; Jäättelá, M.; Nylandsted, J. Methods for the quantification of lysosomal membrane permeabilization: a hallmark of lysosomal cell death. Methods Cell Biol. 2015, 126, 261-285.
De Backer, L.; Braeckmans, K.; Demeester, J.; De Smedt, S. C.; Raemdonck, K. The influence of natural pulmonary surfactant on the efficacy of siRNA-loaded dextran nanogels. Nanomedicine (Lond.) 2013, 8, 1625-1638.
Funk, R. S.; Krise, J. P. Cationic Amphiphilic Drugs Cause a Marked Expansion of Apparent Lysosomal Volume: Implications for an Intracellular Distribution-Based Drug Interaction. Mol. Pharm. 2012, 9, 1384-95.
Kirkegaard, T.; Roth, A. G.; Petersen, N. H.; Mahalka, A. K.; Olsen, O. D.; Moilanen, I.; Zylicz, A.; Knudsen, J.; Sandhoff, K.; Arenz, C., et al. Hsp70 Stabilizes Lysosomes and Reverts Niemann-Pick Disease-Associated Lysosomal Pathology. Nature 2010, 463, 549-53.
Kornhuber, J.; Tripal, P.; Reichel, M.; Terfloth, L.; Bleich, S.; Wiltfang, J.; Gulbins, E. Identification of new functional inhibitors of acid sphingomyelinase using a structure-property-activity relation model. J. Med. Chem. 2008, 51, 219-37.
Kornhuber, J.; Tripal, P.; Reichel, M.; Muhle, C.; Rhein, C.; Muehlbacher, M.; Groemer, T. W.; Gulbins, E. Functional Inhibitors of Acid Sphingomyelinase (FIASMAs): a novel pharmacological group of drugs with broad clinical applications. Cell Physiol. Biochem. 2010, 26, 9-20.
Petersen, N. H.; Olsen, O. D.; Groth-Pedersen, L.; Ellegaard, A. M.; Bilgin, M.; Redmer, S.; Ostenfeld, M. S.; Ulanet, D.; Dovmark, T. H.; Lonborg, A., et al. Transformation-Associated Changes in Sphingolipid Metabolism Sensitize Cells to Lysosomal Cell Death Induced by Inhibitors of Acid Sphingomyelinase. Cancer Cell 2013, 24, 379-93.
Rehman, Z. U.; Hoekstra, D.; Zuhorn, I. S. Mechanism of Polyplex- and Lipoplex-Mediated Delivery of Nucleic Acids: Real-Time Visualization of Transient Membrane Destabilization without Endosomal Lysis. ACS Nano 2013, 7, 3767-3777.
Shoemaker, C. J.; Schornberg, K. L.; Delos, S. E.; Scully, C.; Pajouhesh, H.; Olinger, G. G.; Johansen, L. M.; White, J. M. Multiple Cationic Amphiphiles Induce a Niemann-Pick C Phenotype and Inhibit Ebola Virus Entry and Infection. Plos One 2013, 8, e56265.
te Vruchte, D.; Speak, A. O.; Wallom, K. L.; Al Eisa, N.; Smith, D. A.; Hendriksz, C. J.; Simmons, L.; Lachmann, R. H.; Cousins, A.; Hartung, R., et al. Relative Acidic Compartment Volume as a Lysosomal Storage Disorder-Associated Biomarker. J. Clin. Invest. 2014, 124, 1320-1328.
Wishart DS, Knox C, Guo AC, Shrivastava S, Hassanali M, Stothard P, Chang Z, Woolsey J. DrugBank: a comprehensive resource for in silico drug discovery and exploration. Nucleic Acids Res. 2006 Jan. 1; 34.
Zhang H, De Smedt SC, Remaut K. Fluorescence Correlation Spectroscopy to find the critical balance between extracellular association and intracellular dissociation of mRNA complexes. Acta Biomater. 2018 Jul. 15; 75:358-370.
Joris, F.; De Backer, L.; Van de Vyver, T.; Bastiancich, C.; De Smedt, S. C.; Raemdonck, K. Repurposing Cationic Amphiphilic Drugs as Adjuvants to Induce Lysosomal SiRNA Escape in Nanogel Transfected Cells. J. Control. Release 2018, 269, 266-276.
Van de Vyver, T.; Bogaert, B.; De Backer, L.; Joris, F.; Guagliardo, R.; Van Hoeck, J.; Merckx, P.; Van Calenbergh, S.; Ramishetti, S.; Peer, D.; et al. Cationic Amphiphilic Drugs Boost the Lysosomal Escape of Small Nucleic Acid Therapeutics in a Nanocarrier-Dependent Manner. ACS Nano 2020, 14, 4774-4791.
MacLachlan, I. Liposomal Formulations for Nucleic Acid Delivery. Antisense Drug Technol. Princ. Strateg. Appl. Second Ed. 2007, 237-270.
Buyens, K.; De Smedt, S. C.; Braeckmans, K.; Demeester, J.; Peeters, L.; Van Grunsven, L. A.; De Mollerat Du Jeu, X.; Sawant, R.; Torchilin, V.; Farkasova, K.; et al. Liposome Based Systems for Systemic SiRNA Delivery: Stability in Blood Sets the Requirements for Optimal Carrier Design. J. Control. Release 2012, 158, 362-370.
Meisel, J. W.; Gokel, G. W. A Simplified Direct Lipid Mixing Lipoplex Preparation: Comparison of Liposomal-, Dimethylsulfoxide-, and Ethanol-Based Methods. Sci. Rep. 2016, 6, 1-12.
Raes, L.; Stremersch, S.; Fraire, J. C.; Brans, T.; Goetgeluk, G.; De Munter, S.; Van Hoecke, L.; Verbeke, R.; Van Hoeck, J.; Xiong, R.; et al. Intracellular Delivery of MRNA in Adherent and Suspension Cells by Vapor Nanobubble Photoporation. Nano-Micro Lett. 2020, 12.

1. A nanoparticle comprising at least one lipid, at least one cationic amphiphilic compound (CAC) having a c log P value of less than 10, and at least one nucleic acid; wherein said CAC comprises at least one cyclic moiety.

2. The nanoparticle according to claim 1, wherein said nanoparticle does not comprise a cationic or ionizable lipid.

3. The nanoparticle according to claim 1, wherein said at least one lipid is selected from the group consisting of an ionizable lipid, cationic lipid, a phospholipid, a sterol, sterol analogue, sterol-modified lipid, cholesterol lipid, a PEGylated lipid, a sphingolipid, lysolipid, mixed acyl lipid, ether lipid, ester lipid, oxidized lipid, neutral lipid, zwitterionic lipid, charged lipid, natural lipid, glycosylated lipid, pH-sensitive lipid, isoprenoids, bacterial lipid, plant lipid, bioactive lipid, lipid adjuvants, coenzyme A modified lipid, photo switchable lipids, click lipids, bile lipid, headgroup-modified lipid, fatty acid modified lipids, inverted headgroup lipid, polymer-conjugated lipid, polymerizable lipid, stabilizing lipid, and any combination thereof.

4. The nanoparticle according to claim 3, wherein said at least one lipid is a cationic or ionizable lipid.

5. The nanoparticle according to claim 1, comprising about 5 to about 75 mole percent of said cationic amphiphilic compound.

6. The nanoparticle according to claim 1, wherein said nanoparticle comprises about 5 to about 75 mole percent of said at least one lipid.

7. The nanoparticle according to claim 1, wherein the nucleic acid molecule is selected from the group consisting of DNA, RNA, hybrids of DNA or RNA, RNAi-inducing agents, RNAi agents, antisense RNAs, ribozymes, catalytic DNA, circular RNA, guide RNA, RNAs that induce triple helix formation, aptamers, and vectors.

8. The nanoparticle according to claim 7, wherein the RNA is selected from the group consisting of an antisense compound, messenger RNA (mRNA), self-amplifying mRNA, short interfering nucleic acid (siNA), small interfering RNA (siRNA), asymmetrical interfering RNA (aiRNA), double stranded RNA (dsRNA), micro-RNA (miRNA), small nucleolar RNA (sno-RNA), Piwi-interacting RNA (piRNA), non-coding RNA (ncRNA) and short hairpin RNA (shRNA).

9. The nanoparticle according to claim 1, comprising a lipid: mRNA weight ratio of about 2:1 to about 75:1.

10. The nanoparticle according to claim 1, wherein said CAC is a tricyclic compound.

11. The nanoparticle according to claim 1, wherein said CAC is represented by formula I 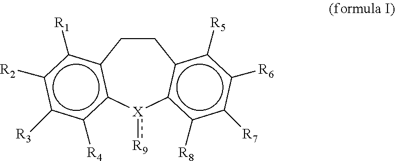
wherein:
R₁, R₂, R₃, R₄, R₅, R₆, R₇ and R₈ are each independently selected from the group consisting of —H, —C₁₋₆alkyl and -halo;
R₉ is selected from group consisting of —(C₁₋₈alkyl)NR₁₀R₁₁, -Het₁;
R₁₀ and R₁₁ are each independently selected from the group consisting of —H and C₁₋₆alkyl;
Het₁ is a 5 or 6-membered heterocycle having from 1 to 3 heteroatoms selected from N, O and S;
X is selected from C, CH and N; and [symbol]
represents a single or double bond, wherein when X is C, then [symbol] represents a double bond.

12. The nanoparticle according to claim 1, wherein said at least one cationic amphiphilic compound is selected from the group consisting of

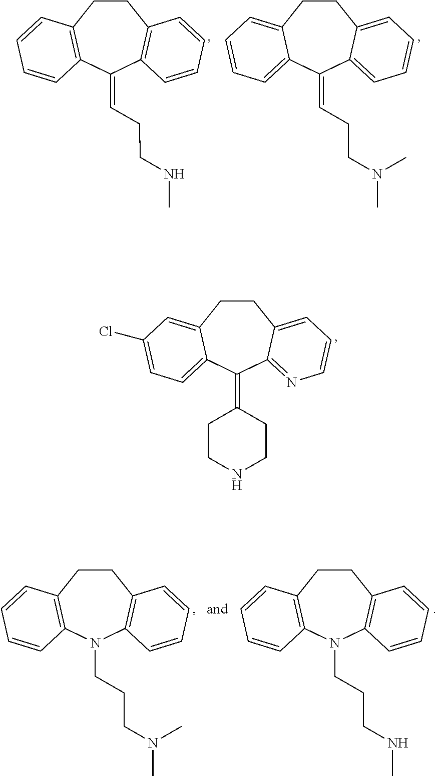

13. (canceled)

14. (canceled)

15. A method for delivery of an active agent across a cell membrane, said method comprising contacting cells with the nanoparticle according to claim 1.

16. The method according to claim 15, wherein the method is carried out in vitro, ex vivo, or in vivo.

17. The method according to claim 16, wherein the cells are human or animal cells.

18. The nanoparticle according to claim 4, comprising about 5 to about 75 mole percent of said cationic or ionizable lipid.

19. The nanoparticle according to claim 5, comprising about 25 to about 75 mole percent of said cationic amphiphilic compound.

20. The nanoparticle according to claim 9, wherein the lipid:mRNA weight ratio is from about 5:1 to about 50:1.

21. The nanoparticle according to claim 20, wherein the lipid:mRNA weight ratio is from about 10:1 to about 30:1.

22. The nanoparticle according to claim 21, wherein the lipid:mRNA weight ratio is about 10:1.
